(19) United States
(12) Patent Application Publication
(10) Pub. No.: US 2025/0010285 A1
(43) Pub. Date: Jan. 9, 2025

(54) FLOW CELLS WITH DENDRON ARCHITECTURE

(71) Applicant: ILLUMINA, INC., San Diego, CA (US)

(72) Inventors: Wayne N. George, Ilford (GB); Iuliana Petruta Maria, Cambridge (GB); Randall Smith, San Diego, CA (US); Andrew A. Brown, Cambridge (GB)

(21) Appl. No.: 18/750,789

(22) Filed: Jun. 21, 2024

Related U.S. Application Data
(60) Provisional application No. 63/511,458, filed on Jun. 30, 2023.

(51) Int. Cl. B01L 3/00; C07D 257/08; C07F 9/6561

(52) U.S. Cl. CPC B01L 3/502; C07D 257/08; C07F 9/6561; B01L 2200/12; B01L 2300/16

(57) ABSTRACT

An example of a flow cell includes a substrate including a surface and a dendron architecture. The dendron architecture includes a functionalized focal point of attachment that is attached to the substrate surface and a plurality of peripheral functional groups that are orthogonal to the functionalized focal point of attachment. The flow cell further includes a primer set attached to the dendron architecture via the plurality of peripheral functional groups.

### CROSS-REFERENCE TO RELATED APPLICATIONS

This application claims the benefit of U.S. Provisional Application Ser. No. 63/511,458, filed Jun. 30, 2023, the content of which is incorporated by reference herein in its entirety.

### REFERENCE TO SEQUENCE LISTING

The Sequence Listing submitted herewith is hereby incorporated by reference in its entirety. The name of the file is ILI264B_IP-2640-US_Sequence_Listing.xml, the size of the file is 14,908 bytes, and the date of creation of the file is Jun. 21, 2024.

### BACKGROUND

Some available platforms for sequencing nucleic acids and other biomolecules utilize a sequencing-by-synthesis approach. With this approach, a nascent strand is synthesized, and the addition of each monomer (e.g., nucleotide) to the growing strand is detected optically and/or electronically. As a template strand directs synthesis of the nascent strand, one can infer the sequence of the template DNA from the series of nucleotide monomers that were added to the growing strand during the synthesis process. In some examples of sequencing-by-synthesis, sequential paired-end sequencing may be used, where forward strands are sequenced and removed, and then reverse strands are constructed and sequenced.

### SUMMARY

Examples of the flow cells and methods disclosed herein utilize a substrate including a surface, where the substrate surface has a dendron architecture attached thereto. The dendron architecture includes a plurality of peripheral groups, where each peripheral group is capable of forming a chemical bond with an oligonucleotide primer. Because each peripheral functional group is chemically available for oligonucleotide primer attachment, a concentration of the primers utilized in the flow cell (relative to flow cell surface area) may be controlled to enhance signal strength during sequencing operations.

In some examples, the flow cells and methods further utilize a regenerating moiety that enables the flow cell to be used multiple times. As such, these flow cells further enable sequencing operations to be performed with resource efficiency.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a top view of an example of a flow cell;

FIG. 1B is an enlarged, perspective, and partially cutaway view of an example of an architecture within a flow channel of the flow cell;

FIG. 1C is an enlarged, perspective, and partially cutaway view of another example of an architecture within a flow channel of the flow cell;

FIG. 1D is an enlarged, perspective, and partially cutaway view of still another example of an architecture within a flow channel of the flow cell;

FIG. 2A through FIG. 2D are schematic views that together illustrate an example of a method disclosed herein, where FIG. 2A depicts the formation of a depression in a resin layer of a substrate, FIG. 2B depicts the application of a polymeric hydrogel over the structure of FIG. 2A, FIG. 2C depicts polishing of the polymeric hydrogel from interstitial regions of the structure of FIG. 2B, and FIG. 2D depicts the introduction of a dendron architecture including pre-grafted primers to a surface of the polymeric hydrogel within the depression;

FIG. 3A through FIG. 3F are schematic views that together illustrate an example of another method disclosed herein, where FIG. 3A depicts the formation of a depression in a resin layer, FIG. 3B depicts the application of a sacrificial layer over the structure of FIG. 3A, FIG. 3C depicts the removal of the sacrificial layer from within the depression, FIG. 3D depicts the application of a polymeric hydrogel over the structure of FIG. 3C, FIG. 3E depicts removing remaining sacrificial layer material from interstitial regions of the structure of FIG. 3D, and FIG. 3F depicts introducing a dendron architecture including pre-grafted primers to a surface of the polymeric hydrogel.

FIG. 4 is schematic flow diagram that illustrates two examples of yet another method disclosed herein, where “A” depicts the formation of a depression in a resin layer, “B” depicts introducing a dendron architecture to a surface of the resin layer within the depression, and “C” depicts primers that are grafted to peripheral groups of the dendron architecture;

FIG. 5 is a graphical representation of the results of fluorescence intensity measurements of a flow cell including a synthesized dendron architecture disclosed herein versus a flow cell including a comparative dendron architecture, with incubation and dendritic conditions of each flow cell lane being shown on the x axis, and normalized median intensity (in arbitrary units) being shown on the y axis;

FIG. 6 is a graphical representation of the results of fluorescence intensity measurements of different lanes of a flow cell, with oligo concentration and dendritic conditions for each lane of the flow cell being shown on the x axis, and normalized median intensity (in arbitrary units) being shown on the y axis;

FIG. 7A is a graphical representation of the results of fluorescence intensity measurements of different lanes of a flow cell, with primer type and concentration being shown on the x axis, and normalized median intensity (in arbitrary units) being shown on the y axis;

FIG. 7B is a graphical representation of the results of primer density determined from the results in FIG. 7A, with primer type and concentration being shown on the x axis, and primer density (in primers per square millimeter) being shown on the y axis;

FIG. 8A and FIG. 8B respectively illustrate the read 1 (R1) fluorescence intensity (FIG. 8A, y axis) and the read 2 (R2) fluorescence intensities (FIG. 8B, y axis) for the red channel after one sequencing cycle (C1) as a function of the CFR intensity (x axis, from FIG. 7A);

FIG. 9 includes three graphs, respectively depicting the error rate (%, y axis, bottom), the percentage of aligned reads (Aligned %, y axis, middle), and the Q30 score (%>=Q30, y axis, top) for first and second reads generated using a flow cell with different primer type and/or concentration (both of which are shown on the x axis); and

FIG. 10 is a graph depicting cluster signal metrics extracted using an offline analysis.

### DETAILED DESCRIPTION

Examples of the flow cells disclosed herein may be used for sequencing processes, examples of which include sequential paired-end nucleic acid sequencing. During an example of sequential paired-end sequencing, individual primers (included as part of a primer set) become attached to peripheral groups of a dendron architecture that is anchored to a flow cell surface. The primers in the primer set include orthogonal cleaving (linearization) chemistry that enables forward strands to be generated, sequenced, and then removed, and then enables reverse strands to be generated, sequenced, and then removed. In these examples, orthogonal cleaving chemistry may be realized through different cleavage sites that are attached to the different primers in the set.

Several example methods are described herein to generate flow cells including a dendron architecture.

### Definitions

It is to be understood that terms used herein will take on their ordinary meaning in the relevant art unless specified otherwise. Several terms used herein and their meanings are set forth below.

An “acrylamide monomer,” as used herein, refers to a monomer with the structure

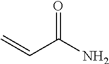

or a monomer including an acrylamide group. Examples of the monomer including an acrylamide group include azido acetamido pentyl acrylamide:

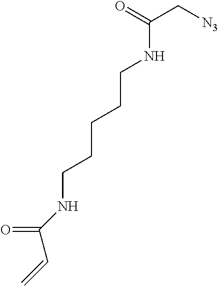 [SMILES: C=CC(=O)NCCCCCNC(=O)CN=[N+]=[N-]]

### and N-isopropylacrylamide:

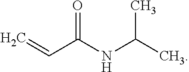

Other acrylamide monomers may be used.

An “acrylate,” as used herein, refers to H₂C═CHCO₂_{^{−}}.

An “activated ester,” as used herein, refers to an ester functional group that has been modified, e.g., with an electronegative substituent, to render the ester susceptible to interaction with a nucleophile.

An “aldehyde,” as used herein, refers to an organic compound containing a functional group with the structure-CHO, which includes a carbonyl center (i.e., a carbon double-bonded to oxygen) with the carbon atom also bonded to hydrogen and an R group, such as an alkyl or other side chain. The general structure of an aldehyde is:

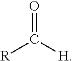

“Alkenyl,” as used herein, refers to a straight or branched hydrocarbon chain containing one or more double bonds. The alkenyl group may have 2 to 20 carbon atoms. Example alkenyl groups include ethenyl, propenyl, butenyl, pentenyl, hexenyl, and the like.

“Alkyl,” as used herein, refers to a straight or branched hydrocarbon chain that is fully saturated (i.e., contains no double or triple bonds). The alkyl group may have 1 to 20 carbon atoms. Example alkyl groups include methyl, ethyl, propyl, isopropyl, butyl, isobutyl, tertiary butyl, pentyl, hexyl, and the like. As an example, the designation “C1-4 alkyl” indicates that there are one to four carbon atoms in the alkyl chain, i.e., the alkyl chain is selected from the group consisting of methyl, ethyl, propyl, iso-propyl, n-butyl, isobutyl, sec-butyl, and t-butyl.

“Alkyne” or “alkynyl,” as used herein, refer to a straight or branched hydrocarbon chain containing one or more triple bonds. The alkynyl group may have 2 to 20 carbon atoms.

An “amine” or “amino” functional group, as used herein, refers to an —NRₐR_{b} group, where Rₐ and R_{b} are each independently selected from hydrogen

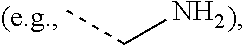

C1-6 alkyl, C2-6 alkenyl, C2-6 alkynyl, C3-7 carbocycle, C6-10 aryl, 5-10 membered heteroaryl, and 5-10 membered heterocyclyl, as defined herein.

“Aryl,” as used herein, refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms. Examples of aryl groups include phenyl, naphthyl, azulenyl, and anthracenyl.

“Aryl fluorosulfate,” as used herein, refers to an aryl group that includes a fluorosulfate substituent.

An “aryl azide” functional group refers to an aryl group that includes an azide substituent.

The term “attached,” as used herein, refers to the state of two things being joined, fastened, adhered, connected or bound to each other, either directly or indirectly. For example, a functional material (e.g., a polymeric hydrogel, an oligonucleotide primer, etc.) can be attached to a target surface (e.g., of a resin layer) by a covalent or non-covalent bond. A covalent bond is characterized by the sharing of pairs of electrons between atoms. A non-covalent bond is a physical bond that does not involve the sharing of pairs of electrons and can include, for example, hydrogen bonds, ionic bonds, Van der Waals forces, hydrophilic interactions and hydrophobic interactions.

An “azide” or “azido” functional group refers to —N₃.

The term “base support,” as used herein, refers to a material upon which another material or material layer (e.g., resin layer) may be introduced. In some instances, the base support is part of a single-layer substrate. In other instances, the base support is part of a multi-layer substrate and has an additional layer (e.g., resin) applied thereon.

A “bonding region,” as used herein, refers to an area of a patterned or unpatterned substrate that is to be bonded to another material, which may be, as examples, a lid, another patterned or unpatterned substrate, etc. The bond that is formed at the bonding region may be a chemical bond (as described above in regard to attachment), or a mechanical bond (e.g., using a fastener, etc.).

The term “carbocycle,” as used herein, means a non-aromatic cyclic ring or ring system containing only carbon atoms in the ring system backbone. When the carbocycle is a ring system, two or more rings may be joined together in a fused, bridged or spiro-connected fashion. Carbocycles may have any degree of saturation, provided that at least one ring in a ring system is not aromatic. Thus, carbocycles include cycloalkyls, cycloalkenyls, and cycloalkynyls. The carbocycle group may have 3 to 20 carbon atoms. Examples of carbocycle rings include cyclopropyl, cyclobutyl, cyclopentyl, cyclohexyl, cyclohexenyl, 2,3-dihydro-indene, bicyclo[2.2.2]octanyl, adamantyl, and spiro[4.4]nonanyl.

The terms “carboxylic acid” or “carboxyl,” as used herein, refer to —COOH.

“Cycloalkylene,” as used herein, refers to a fully saturated carbocycle ring or ring system that is attached to the rest of the molecule via two points of attachment.

“Cycloalkenyl” or “cycloalkane,” as used herein, refer to a carbocycle ring or ring system having at least one double bond, wherein no ring in the ring system is aromatic. Examples include cyclohexenyl or cyclohexene and norbornenyl or norbornene. Also as used herein, “heterocycloalkenyl” or “heterocycloalkene” means a carbocycle ring or ring system with at least one heteroatom in ring backbone, having at least one double bond, wherein no ring in the ring system is aromatic.

“Cycloalkynyl” or “cycloalkyne,” as used herein, refer to a carbocycle ring or ring system having at least one triple bond, wherein no ring in the ring system is aromatic. An example is cyclooctyne (see below). Also as used herein, “heterocycloalkynyl” or “heterocycloalkyne” means a carbocycle ring or ring system with at least one heteroatom in ring backbone, having at least one triple bond, wherein no ring in the ring system is aromatic.

“Cyclooctene” or “cyclooctenyl” refers to an eight-membered ring structure including an alkene bond between two of the carbons in the ring. A “cyclooctene based molecule” refers to a cyclooctene or a derivative thereof (e.g., cis or trans cyclooctene).

“Cyclooctyne” or “cyclooctynyl” refers to an eight-membered ring structure including an alkyne bond between two of the carbons in the ring. A “cyclooctyne based molecule” refers to a cyclooctyne or a derivative thereof. Examples of cyclooctyne derivatives are bicyclononyne (BCN) and dibenzocyclooctyne (DBCO).

A “dendron” or “dendron architecture” or “dendron structure,” as used herein, refers to a branched molecule having a chemically addressable group at one end of the molecule (referred to herein as the functionalized focal point of attachment or focal point) and a plurality of peripheral functional groups located at the terminus of each branch extending from the focal point.

The term “depositing,” as used herein, refers to any suitable application technique, which may be manual or automated, and, in some instances, results in modification of surface properties. Generally, depositing may be performed using vapor deposition techniques, coating techniques, grafting techniques, or the like. Some specific examples include spray coating (e.g., ultrasonic spray coating), spin coating, dunk or dip coating, doctor blade coating, puddle dispensing, flow through coating, aerosol printing, screen printing, microcontact printing, inkjet printing, or the like.

The term “depression,” as used herein, refers to a discrete concave feature in a patterned substrate having a surface opening. The depression may be at least partially surrounded by interstitial region(s) of the patterned substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc. As an example, the depression may be a well or two interconnected wells. The depression may also have more complex architectures, such as ridges, step features, etc.

The term “each,” when used in reference to a collection of items, is intended to identify an individual item in the collection, but does not necessarily refer to every item in the collection. Exceptions can occur if explicit disclosure or context clearly dictates otherwise.

The term “epoxy,” as used herein, refers to

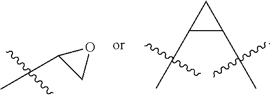

The term “flow cell,” as used herein, is intended to mean a vessel having a flow channel where a reaction can be carried out, an inlet for delivering reagent(s) to the flow channel, and an outlet for removing reagent(s) from the flow channel. In some examples, the flow cell also enables the detection of the reaction that occurs in the flow cell. For example, the flow cell can include one or more transparent surfaces allowing for the optical detection of arrays, optically labeled molecules, or the like.

A “flow channel” or “channel” refers to an area defined between two bonded components, which can selectively receive a liquid sample. In some examples, the flow channel may be defined between a patterned or unpatterned substrate and a lid, and thus may be in fluid communication with one or more surface chemistries on the patterned or unpatterned substrate. In other examples, the flow channel may be defined between two patterned or unpatterned substrates (each of which has surface chemistry thereon), and thus may be in fluid communication with the surface chemistry of the substrates.

The term “functionalized focal point of attachment” is used interchangeably with the term “focal point” herein, and refers to a chemically addressable group included in the chemical structure of a dendron architecture, where the focal point is capable of attaching to a surface of the substrate or to a polymeric hydrogel applied thereon.

“Heteroaryl,” as used herein, refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members.

“Heterocycle,” as used herein, refers to a non-aromatic cyclic ring or ring system containing at least one heteroatom in the ring backbone. Heterocycles may be joined together in a fused, bridged or spiro-connected fashion. Heterocycles may have any degree of saturation provided that at least one ring in the ring system is not aromatic. In the ring system, the heteroatom(s) may be present in either a non-aromatic or aromatic ring. The heterocycle group may have 3 to 20 ring members (i.e., the number of atoms making up the ring backbone, including carbon atoms and heteroatoms). In some examples, the heteroatom(s) are O, N, or S.

The terms “hydrazine” or “hydrazinyl,” as used herein, refer to a —NHNH₂ group.

The terms “hydrazone” or “hydrazonyl,” as used herein, refer to a

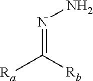

group in which Rₐ and R_{b} are each independently selected from hydrogen, C1-6 alkyl, C2-6 alkenyl, C2-6 alkynyl, C3-7 carbocycle, C6-10 aryl, 5-10 membered heteroaryl, and 5-10 membered heterocycle, as defined herein.

“Hydroxy” or “hydroxyl,” as used herein, refers to an —OH group.

“Nitrile oxide,” as used herein, means a “RₐC≡N⁺O⁻” group in which Rₐ is defined herein. Examples of preparing nitrile oxide include in situ generation from aldoximes by treatment with chloramide-T or through action of base on imidoyl chlorides [RC(Cl)═NOH] or from the reaction between hydroxylamine and an aldehyde.

“Nitrone,” as used herein, refers to a

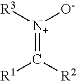

group in which R¹, R², and R³ may be any of the Rₐ and R_{b} groups defined herein, except that R³ is not hydrogen (H).

“Norbornene” or “oxynorbornene,” as used herein, refers to a strained, bridged, cyclic hydrocarbon including a cyclohexene ring and a methylene bridge between C1 and C4.

A “nucleotide,” as used herein, refers to a molecule including a nitrogen-containing heterocyclic base, a sugar, and one or more phosphate groups. Nucleotides are monomeric units of a nucleic acid sequence. In RNA, the sugar is a ribose, and in DNA, the sugar is a deoxyribose, i.e. a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to the N-1 atom of a pyrimidine or to the N-9 atom of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

An “open wafer” refers to the patterned or unpatterned structure which has the surface chemistry applied thereto but does not have a lid or second patterned or unpatterned structure applied thereto.

The term “orthogonal,” when used to describe two functional groups or two cleaving chemistries, means that the groups or chemistries are different from each other. Orthogonal functional groups, such as the focal point and the peripheral groups of the same dendron architecture, are capable of reacting with different functional groups, e.g., an azide may be reacted with an alkyne or DBCO (dibenzocyclooctyne) while an amino may be reacted with an activated carboxylate group or an N-hydroxysuccinimide (NHS) ester. Orthogonal cleaving chemistries are susceptible to different cleaving agents so that the first cleaving chemistry is unaffected when exposed to the cleaving agent for the second cleaving chemistry, and the second cleaving chemistry is unaffected when exposed to the cleaving agent for the first cleaving chemistry.

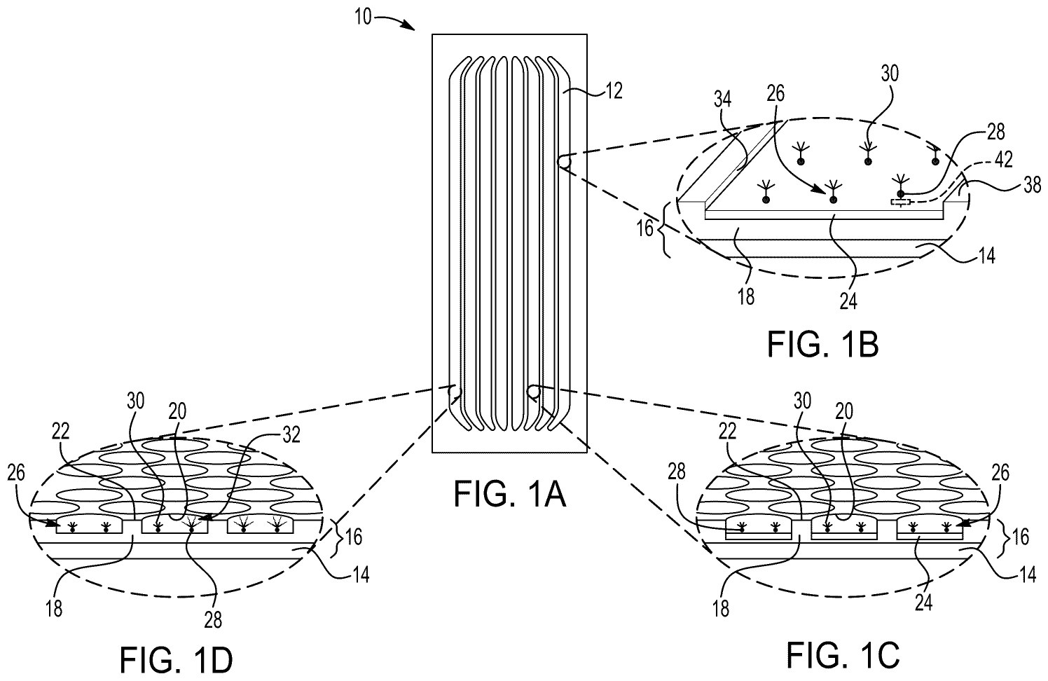

In some examples, the term “over” may mean that one component or material is positioned directly on another component or material. When one component is directly on another, the two are in contact with each other, with no intervening layer or material therebetween. In FIG. 1D, for example, the dendron architecture 26 is shown as being applied directly over the layer 18, without any intervening layer or material therebetween. In other examples, the term “over” may mean that one component or material is positioned indirectly on another component or material. By indirectly on, it is meant that a gap or an additional component or material may be positioned between the two components or materials. In FIG. 1C, for example, the dendron architecture 26 is shown as being applied indirectly over the resin layer 18. The polymeric hydrogel 24 is positioned therebetween.

The term “peripheral functional group” is used interchangeably with the term “peripheral group(s)” herein, and refers to a functional group located at the terminus of a branch of a dendron structure. The peripheral group is capable of grafting a primer thereto.

As used herein, the term “polyhedral oligomeric silsesquioxane” refers to a chemical composition that is a hybrid intermediate (e.g., RSiO_{1.5}) between that of silica (SiO₂) and silicone (R₂SiO). An example of polyhedral oligomeric silsesquioxane may be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups.

A “primer,” as used herein, refers to a single stranded nucleic acid sequence (e.g., single stranded DNA). Some primers serve as a starting point for template amplification and cluster generation. The 5′ terminus of these primers may be modified to allow a coupling reaction with a peripheral functional group of a dendron architecture. Other primers serve as a starting point for DNA synthesis. The primer length can be any number of bases long and can include a variety of non-natural nucleotides. In an example, the sequencing primer is a short strand, ranging from 10 to 60 bases, or from 20 to 40 bases.

A “substrate” refers to a structure that supports surface chemistry, which may be present on the structure in one or more patterns. The substrate may be a single-layer substrate including a base support, or a multi-layer substrate including the base support with a layer (e.g., resin layer) applied thereon (as shown in FIG. 1B, FIG. 1C, and FIG. 1D). In some of the examples disclosed herein, the substrate may be exposed to patterning techniques (e.g., stamping, etching, lithography, etc.) to generate the patterns/architectures for the surface chemistry. The pattern defined in some examples of the substrate may be generated via any of the methods disclosed hereinbelow.

“Sulfonyl fluoride,” as used herein, refers to a compound including —SO₂F that is capable of undergoing a sulfur (VI) fluoride exchange reaction.

“Surface chemistry,” as used herein, refers to i) primers that are, or are to be, attached to a substrate surface (e.g., via a dendron architecture) and that are capable of amplifying a library template strand, or ii) the primers attached to the substrate surface (e.g., via the dendron architecture) and the polymeric hydrogel that attaches the dendron architecture to the substrate. As is described in more detail herein, the surface chemistry may further include the chemistry added to activate a substrate surface, such as a silane or hydroxyl groups.

A “terminal alkene,” as used herein, refers to an alkene in which a carbon-carbon pi bond is located at the end of a carbon chain.

The terms “tetrazine” and “tetrazinyl,” as used herein, refer to six-membered heteroaryl group comprising four nitrogen atoms and having a molecular formula C₂H₂N₄. Tetrazine can be optionally substituted.

“Tetrazole,” as used herein, refers to a five-membered heterocyclic group including four nitrogen atoms and having a molecular formula CH₂N₄. Tetrazole can be optionally substituted.

A “thiol” functional group refers to —SH.

The singular forms “a”, “an”, and “the” include plural referents unless the context clearly dictates otherwise.

The terms comprising, including, containing and various forms of these terms are synonymous with each other and are meant to be equally broad.

The terms top, bottom, lower, upper, on, overlie, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, the term(s) are/is meant to encompass minor variations (up to +/−10%) from the stated value.

### Flow Cells

An example of a flow cell for sequential paired-end sequencing generally comprises a substrate having a surface. The substrate surface may be patterned to include architectures for isolating surface chemistry, such as depressions that are separated by interstitial regions. Alternatively, the substrate may be unpatterned. Unpatterned surfaces include a lane defined therein, where the surface chemistry is applied within the lane. In some examples, the flow cell further includes a polymeric hydrogel applied directly over the substrate surface.

The flow cells described herein further include a dendron architecture that is attached to the substrate surface (or attached to the polymeric hydrogel applied over the substrate surface, when the polymeric hydrogel is included). The dendron architecture includes a focal point that is capable of forming a chemical bond with functional groups of the substrate surface, or with functional groups of the polymeric hydrogel. The dendron architecture further includes a plurality of peripheral functional groups (peripheral groups), where each peripheral group is capable of attaching/grafting a primer of a primer set thereto.

As such, disclosed herein is a flow cell, which includes: a substrate including a surface; a dendron architecture including: a functionalized focal point of attachment that is attached to the substrate surface, and a plurality of peripheral groups that are orthogonal to the functionalized focal point of attachment; and a primer set attached to the dendron architecture via the plurality of peripheral functional groups.

One example of the flow cell 10 is shown in FIG. 1A from a top view. As shown in FIG. 1B through FIG. 1D, the substrate 16 includes a base support 14 and a resin layer 18 applied thereon. While these figures depict the substrate 16 as a multi-layered structure including the components shown, it is to be understood that a single-layer structure may be used instead. In these examples, the flow cell 10 includes the base support 14, without the layer 18 applied thereon.

Any of the patterned or unpatterned substrates 16 described herein may be used as an open wafer, i.e., it does not have a lid or second substrate 16 bonded thereto.

Alternatively, and not shown in the figures, the flow cell 10 may include two substrates 16 (each of which may be either patterned or unpatterned) bonded together, or one substrate 16 bonded to a lid.

When used, the lid may be any material that is transparent to an excitation light that is directed toward the flow cell 10 during a sequencing operation. In optical detection systems, the lid may also be transparent to the emissions generated from reaction(s) taking place in the flow cell 10. As examples, the lid may include glass (e.g., borosilicate, fused silica, etc.) or a transparent polymer. A commercially available example of a suitable borosilicate glass is D 263®, available from Schott North America, Inc. Commercially available examples of suitable polymer materials, namely cycloolefin polymers, are the ZEONOR® products available from Zeon Chemicals L.P. In some instances, the lid is shaped to form the top of the flow cell 10, and in other instances, the lid is shaped to form both the top of the flow cell 10, as well as sidewalls of the flow channel 12.

Between the two substrates 16 or the one substrate 16 and the lid is a flow channel 12. The two substrates 16 or the one substrate 16 and the lid may be bonded together via a spacer layer. When the spacer layer is included, each flow channel 12 is defined by a first substrate 16, the spacer layer, and either the lid or a second substrate 16 (lid and/or second substrate 16 not shown).

The example shown in FIG. 1A includes eight flow channels 12. While eight flow channels 12 are shown in FIG. 1A, it is to be understood that any number of flow channels 12 may be included in the flow cell 10 (e.g., a single flow channel 12, four flow channels 12, etc.). Each flow channel 12 may be isolated from each other flow channel 12 so that fluid introduced into a flow channel 12 does not flow into adjacent flow channel(s) 12. Some examples of the fluids introduced into the flow channel 12 may introduce dendron architectures, reaction components (e.g., DNA sample, polymerases, sequencing primers, nucleotides, etc.), washing solutions, deblocking agents, etc. to the flow cell 10.

Each flow channel 12 is in fluid communication with an inlet and an outlet (not shown in FIG. 1A through FIG. 1D). The inlet and outlet of each flow channel 12 may be positioned at opposed ends of the flow channel 12. The inlets and outlets of the respective flow channels 12 may alternatively be positioned anywhere along the length and width of the flow channel 12 that enables desirable fluid flow.

The inlet allows fluids to be introduced into the flow channel 12, and the outlet allows fluid to be extracted from the flow channel 12. Each of the inlets and outlets is fluidly connected to a fluidic control system (including, e.g., reservoirs, pumps, valves, waste containers, and the like) which controls fluid introduction and expulsion.

The flow channel 12 may have any desirable shape. In an example, the flow channel 12 has a substantially rectangular configuration with curved ends (as shown in FIG. 1A). The length of the flow channel 12 depends, in part, upon the size of the substrate 16 (e.g., the resin layer 18 and/or the base support 14 used to form the substrate 16). The width of the flow channel 12 depends, in part, upon the size of the substrate 16, the desired number of flow channels 12, the desired space between adjacent channels 12, and the desired space at a perimeter of the substrate 16. The spaces between channels 12 and at the perimeter may be sufficient for attachment to a lid (not shown) or another substrate 16 (also not shown).

The depth of the flow channel 12 can be as small as a monolayer thick when microcontact, aerosol, or inkjet printing is used to deposit a separate (spacer) material that defines the flow channel 12 walls. For other examples, the depth of the flow channel 12 can be about 1 μm, about 10 μm, about 50 μm, about 100 μm, or more. In an example, the depth may range from about 10 μm to about 100 μm. In another example, the depth may range from about 10 μm to about 30 μm. In still another example, the depth is about 5 μm or less. It is to be understood that the depth of the flow channel 12 may be greater than, less than, or between the values specified above.

The spacer layer used to attach the substrate 16 and the lid (or used to attach a first substrate 16 and the second substrate 16) may be any material that will seal portions of the substrate 16 and the lid (or that will seal portions of the first substrate 16 and the second substrate 16). As examples, the spacer layer may be an adhesive, a radiation-absorbing material that aids in bonding, or the like. In some examples, the spacer layer is the radiation-absorbing material, e.g., KAPTON® black.

The (first) substrate 16 includes a bonding region where it can be sealed to the lid or to the second substrate 16. The bonding region may be located at the perimeter of the flow channel(s) 12 and at the perimeter of the flow cell 10.

In some examples, the flow channel 12 is at least partially defined by the resin layer 18 of at least one patterned substrate 16 (as shown in FIG. 1C and FIG. 1D). The patterned substrate 16, when used, includes the base support 14 and the resin layer 18 having depressions 20 defined therein, where the depressions 20 are separated from one another by interstitial regions 22. When a single-layer substrate is used, the base support 14 may be patterned to include depressions 20, as no layer 18 is included.

In other examples, the flow channel 12 is at least partially defined by the resin layer 18 of at least one unpatterned substrate 16 (as shown in FIG. 1B). The unpatterned substrate 16, when used, includes the resin layer 18 applied directly over the base support 14, without any depressions 20 defined in the resin layer 18. In the unpatterned substrate 16, a lane 34 may be defined in the resin layer 18. The unpatterned substrate 16 may alternatively be a single-layer substrate including the base support 14 and the lane 34 defined in the base support 14, without the resin layer 18.

Suitable example materials for the base support 14 are selected to be transparent to the excitation light that is directed toward the flow cell 10 during a sequencing operation. As some examples, the base support 14 may include siloxanes, glass, modified or functionalized glass, polymeric materials (including acrylics, polystyrene and copolymers of styrene and other materials, polyethylene terephthalate (PET), polycarbonate, cyclic olefin copolymer (COC), some polyamides), silica or silicon oxide (e.g., SiO₂), fused silica, silica-based materials, silicon nitride (Si₃N₄), resins, or the like. The material of the base support 14 may be a pure material, a material with some impurities, or a mixture of materials, with the understanding that the resulting base support 14 is capable of the desired transmittance.

The base support 14 may be a circular sheet, a panel, a wafer, a die, etc. having a diameter ranging from about 2 mm to about 300 mm, e.g., from about 200 mm to about 300 mm, or may be a rectangular sheet, panel, wafer, die etc. having its largest dimension up to about 10 feet (˜3 meters). For example, a die may have a width ranging from about 0.1 mm to about 10 mm. While example dimensions have been provided, it is to be understood that a base support 14 with any suitable dimensions to support the resin layer 18 may be used.

As described, examples of the patterned or unpatterned substrate 16 include the resin layer 18 applied over the base support 14. The resin layer 18 may include a material that can be etched or imprinted to form a lane 34 (FIG. 1B) or depressions 20 (FIG. 1C and FIG. 1D). Examples of suitable materials for the resin layer 18 include polymeric resins, such as a polyhedral oligomeric silsesquioxane based resin (e.g., POSS® from Hybrid Plastics), a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As will be described in more detail herein, the material of the resin layer 18 may be selected to include functional groups that are suitable for attaching the focal point of the dendron architecture thereto, or may be functionalized to introduce such groups to the resin layer 18.

Suitable deposition techniques for the resin layer 18 include dip coating, dunk coating, spin coating, spray coating, puddle dispensing, ultrasonic spray coating, doctor blade coating, aerosol printing, screen printing, microcontact printing, etc.

As described, the resin layer 18 may be processed to form the lane 34 surrounded by a bonding region 38 (as shown in FIG. 1B) or to form the depressions 20 separated by interstitial regions 22 (as shown in FIG. 1C and FIG. 1D). Suitable patterning techniques for the layer 18 include photolithography, nanoimprint lithography (NIL), stamping techniques, embossing techniques, molding techniques, microetching techniques, etc., some of which are described in more detail herein. The deposition and patterning techniques that are used may depend, in part, upon the material used for the base support 14 and the material used for the resin layer 18.

FIG. 1B and FIGS. 1C and 1D depict two different architectures within the flow channel 12 of the flow cell 10. Each of these will now be described.

In FIG. 1B, the substrate 16 (specifically the resin layer 18 of the multi-layered substrate) has a lane 34 defined therein. The lane 34 is a concave region in the substrate 16. The lane 34 may be defined via etching, imprinting, lithography, or another suitable technique.

The lane 34 may have any desirable shape. In an example, the lane 34 has a substantially rectangular configuration with curved ends (similar to the channel outlines shown in FIG. 1A). The length of the lane 34 depends, in part, upon the size of the substrate 16. The width of the lane 34 depends, in part, upon the size of the substrate 16, the desired number of lanes 34, the desired space between adjacent lanes 34, and the desired space at a perimeter of the substrate 16.

The surface of the substrate 16 surrounding the lane(s) 34 may be sufficient for attachment to another substrate 16 or the lid, and thus is a bonding region 38. The other substrate 16 or the lid also includes a surface that is sufficient for attachment to the bonding region 38.

It is to be understood that in the final flow cell 10, the lane 34 (when included) is in fluid communication with the flow channel 12.

In FIG. 1C and FIG. 1D, the substrate 16 (specifically the resin layer 18 of the multi-layered substrate) has depressions 20 defined therein.

Many different layouts of the depressions 20 may be envisaged, including regular, repeating, and non-regular patterns. In an example, the depressions 20 are disposed in a hexagonal grid for close packing and improved density. Other layouts may include, for example, rectilinear (rectangular) layouts, triangular layouts, and so forth. In some examples, the layout or pattern can be an x-y format in rows and columns. In some other examples, the layout or pattern can be a repeating arrangement of the depressions 20 and the interstitial regions 22. In still other examples, the layout or pattern can be a random arrangement of the depressions 20 and the interstitial regions 22.

The layout or pattern may be characterized with respect to the density (number) of the depressions 20 in a defined area. For example, the depressions 20 may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about 50 million per mm², or more, or less. It is to be further understood that the density can be between one of the lower values and one of the upper values selected from the ranges above, or that other densities (outside of the given ranges) may be used. As examples, a high density array may be characterized as having the depressions 20 separated by less than about 100 nm, a medium density array may be characterized as having the depressions 18 separated by about 400 nm to about 1 μm, and a low density array may be characterized as having the depressions 20 separated by greater than about 1 μm.

The layout or pattern of the depressions 20 may also or alternatively be characterized in terms of the average pitch, or the spacing from the center of one depression 20 to the center of an adjacent depression 20, or from the right edge of one depression 20 to the left edge of an adjacent depression 20 (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of depressions 20 can be between one of the lower values and one of the upper values selected from the ranges above. In an example, the depressions 20 have a pitch (center-to-center spacing) of about 1.5 μm. While example average pitch values have been provided, it is to be understood that other average pitch values may be used.

The size of each depression 20 may be characterized by its volume, opening area, depth, and/or diameter or length and width. For example, the volume can range from about 1×10⁻³ μm³ to about 100 μm³, e.g., about 1×10⁻² μm³, about 0.1 μm³, about 1 μm³, about 10 μm³, or more, or less. For another example, the opening area can range from about 1×10⁻³ μm² to about 100 μm², e.g., about 1×10⁻² μm², about 0.1 μm², about 1 μm², at least about 10 μm², or more, or less. For still another example, the depth can range from about 0.1 μm to about 100 μm, e.g., about 0.5 μm, about 1 μm, about 10 μm, or more, or less. For another example, the depth can range from about 0.1 μm to about 100 μm, e.g., about 0.5 μm, about 1 μm, about 10 μm, or more, or less. For yet another example, the diameter or each of the length and the width can range from about 0.1 μm to about 100 μm, e.g., about 0.5 μm, about 1 μm, about 10 μm, or more, or less.

It is to be understood that in the final flow cell 10, the depressions 20 (when included) are in fluid communication with the flow channel 12.

While not shown in FIG. 1C or FIG. 1D, a portion of the surface of the substrate 16 surrounding the depressions 20 may be sufficient for attachment to another substrate 16 or the lid, and thus is a bonding region 38.

As shown in FIG. 1B and FIG. 1C, the resin layer 18 may have the polymeric hydrogel 24 applied within the lane 34 (FIG. 1B) or the depressions 20 (FIG. 1C). As is described in more detail herein, the resin layer 18 may be silanized to facilitate attachment of the polymeric hydrogel 24 attachment thereto. In some examples, such as in the example depicted in FIG. 1D, the polymeric hydrogel 24 is not included in the flow cell 10.

The polymeric hydrogel 24 may be a material that can swell when liquid is taken up and can contract when liquid is removed, e.g., by drying. In one specific example, the polymeric hydrogel includes an acrylamide copolymer, such as poly(N-(5-azidoacetamidylpentyl) acrylamide-co-acrylamide, PAZAM. PAZAM and some other forms of the acrylamide copolymer are represented by the following structure (I):

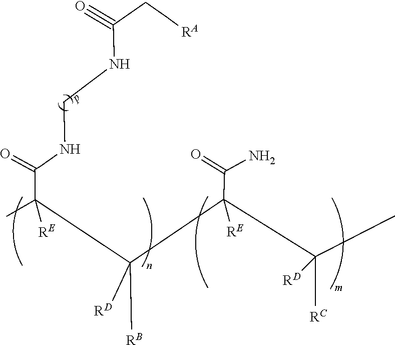

wherein:
R^{A} is selected from the group consisting of azido, optionally substituted amino (e.g., an aminooxy group), optionally substituted alkenyl, optionally substituted alkyne, halogen, optionally substituted hydrazone, optionally substituted hydrazine, carboxyl, hydroxy, optionally substituted tetrazole, optionally substituted tetrazine, nitrile oxide, nitrone, sulfate, and thiol;
R^{B} is H or optionally substituted alkyl;
R^{C}, R^{D}, and R^{E} are each independently selected from the group consisting of H and optionally substituted alkyl;
each of the —(CH₂)ₚ— can be optionally substituted;
p is an integer in the range of 1 to 50;
n is an integer in the range of 1 to 50,000; and
m is an integer in the range of 1 to 100,000.

One of ordinary skill in the art will recognize that the arrangement of the recurring “n” and “m” features in structure (I) are representative, and the monomeric subunits may be present in any order in the polymer structure (e.g., random, block, patterned, or a combination thereof).

The molecular weight of PAZAM and other forms of the acrylamide copolymer may range from about 5 kDa to about 1500 kDa or from about 10 kDa to about 1000 kDa, or may be, in a specific example, about 312 kDa.

In some examples, PAZAM and other forms of the acrylamide copolymer are linear polymers. In some other examples, PAZAM and other forms of the acrylamide copolymer are lightly cross-linked polymers.

In other examples, the polymeric hydrogel 24 may be a variation of the structure (I). In one example, the acrylamide unit may be replaced with N,N-dimethylacrylamide

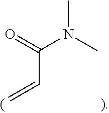

In this example, the acrylamide unit in structure (I) may be replaced with

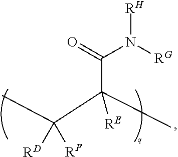

where R^{D}, R^{E}, and R^{F} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

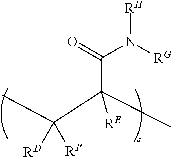

in addition to the recurring “n” and “m” features, where R^{D}, R^{E}, and R^{F} are each H or a C1-C6 alkyl, and R^{G} and R^{H} are each a C1-C6 alkyl. In this example, q may be an integer in the range of 1 to 100,000.

As another example of the polymeric hydrogel 24, the recurring “n” feature in structure (I) may be replaced with a monomer including a heterocyclic azido group having structure (II):

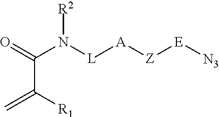

wherein R¹ is H or a C1-C6 alkyl; R² is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the polymeric hydrogel 24 may include a recurring unit of each of structure (III) and (IV):

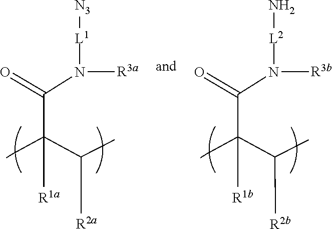

wherein each of R^{1a}, R^{2a}, R^{1b} and R^{2b} is independently selected from hydrogen, an optionally substituted alkyl or optionally substituted phenyl; each of R^{3a} and R^{3b} is independently selected from hydrogen, an optionally substituted alkyl, an optionally substituted phenyl, or an optionally substituted C7-C14 aralkyl; and each L¹ and L² is independently selected from an optionally substituted alkylene linker or an optionally substituted heteroalkylene linker.

The polymeric hydrogel 24 may be prepared by polymerizing the monomer(s) that are to form the hydrogel 24. The polymerization process and process conditions will depend upon the monomer(s) used to form the hydrogel 24. In an example, the hydrogel 24 may be synthesized using reversible addition-fragmentation chain transfer (RAFT) polymerization. While RAFT polymerization may be used, it is to be understood that other polymerization processes may also be used. Other suitable polymerization processes include atom transfer radical polymerization (ATRP), nitroxide mediated radical (NMP) polymerization in combination with RAFT or ATRP, NMP with an additional cross-linking step, cobalt-mediated polymerization, group transfer polymerization (GTP), ring opening polymerization (ROP), ionic polymerization, or any other polymerization process that either directly or indirectly yields the desired linear or branched architecture. In some instances, the polymerization of the polymeric hydrogel 24 may be triggered by exposing the polymeric hydrogel 24 to a UV light (e.g., after the monomeric components of the polymeric hydrogel 24 have been deposited at desired areas of the flow cell 10).

When included, the polymeric hydrogel 24 includes functional groups that are capable of attaching the focal point of the dendron architecture thereto. Specific examples of these functional groups for the polymeric hydrogel 24 are described herein in regard to Table 1.

As shown in FIG. 1B through FIG. 1D, the flow cell 10 includes the dendron architecture 26 attached to the substrate surface via the focal point 28 of the dendron 26. The polymeric hydrogel 24, when used, includes functional groups that attach the focal point 28 of the dendron architecture 26 thereto (as shown in FIG. 1B and FIG. 1C). When the polymeric hydrogel 24 is not included, the resin layer 18 of the multi-layered substrate (or the base support 14 of the single-layer substrate) includes functional groups that attach the focal point 28 thereto (as shown in FIG. 1D). As such, the focal point 28 of the dendron 26 anchors the dendron 26 to the surface of the substrate 16 via the polymeric hydrogel 24 or via the resin layer 18 through chemical attachment.

The focal point 28 of the dendron 26 may be a single chemically addressable focal point 28. While not shown in FIG. 1B through FIG. 1D, in some examples, the dendron 26 has a bowtie structure. In these examples, rather than the single chemically addressable focal point 28, the dendron structure includes a focal point 28 having two or more branching moieties, where each branch terminates in a functional group that is capable of attaching (e.g., bonding) to the resin layer 18 or to the polymeric hydrogel 24 of the flow cell 10. Without being bound by any particular theory, it is believed that utilizing a bowtie structure for the dendron 26 may enhance attachment of the dendron 26 to the substrate surface.

The dendron 26 further includes peripheral groups 30 that are capable of attaching primers of a primer set 32 thereto, as shown in two of the depressions 20 in FIG. 1D. While FIG. 1B through FIG. 1D depict dendron architectures 26 having four peripheral groups 30, it is to be understood that the number of peripheral groups 30 included in each dendron 26 may vary. In some examples of the flow cells 10 described herein, the number of peripheral groups 30 included in the dendron architecture 26 ranges from 2 to 20.

The focal point 28 of the dendron 26 is selected to be orthogonal to the chemical functionality of the peripheral groups 30. The orthogonality of the focal point 28 and the peripheral groups 30 may orient the dendron 26 on the flow cell 10 surface, such that the peripheral groups 30 have no affinity for the resin layer 18 or for the polymeric hydrogel 24 and remain chemically available for attaching primers of the primer set 32 thereto.

The functionalized focal point of attachment 28 may include a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate. In these examples, each of the plurality of peripheral functional groups 30 includes a functional group that is selected from the group consisting of an azide, an aryl azide, an amine, a norbornene, a tetrazole, tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate. As noted however, the chemistries of the focal point 28 and the peripheral functional groups 30 should be orthogonal. It is to be understood that when one of the focal point 28 or the peripheral groups 30 include(s) the cyclooctene based molecule, the focal point 28 or the peripheral groups 30 may be capable of undergoing the inverse electron demand Diels Alder reaction when the other of the peripheral groups 30 or the focal point 28 includes a tetrazine functionality.

In a specific example, the functionalized focal point of attachment 28 is a tetrazine, the plurality of peripheral functional groups 30 is a plurality of azides, and the substrate surface includes a norbornene functional group that attaches the functionalized focal point of attachment 28 to the substrate surface. In another specific example, the functionalized focal point of attachment 28 is an N-hydroxysuccinimide (NHS), the plurality of peripheral groups 30 is a plurality of azides, and the polymeric hydrogel surface includes an amine functional group that attaches the functionalized focal point of attachment 28 to the polymeric hydrogel surface.

Several suitable examples of compatible chemical functionalities that may be used to attach the focal point 28 of the dendron 26 to a desired attachment point on the flow cell 10 surface are set forth in Table 1 below. For each of the given focal points 28, any of the listed attachment points may be used.

TABLE 1

| Dendron Focal | Functionality of Desired Attachment Point | | | |
|---|---|---|---|---|
| Point | (e.g., on resin layer 18 or hydrogel 24) | | | |
| azide/aryl azide | alkyne | norbornene | phenyl- | cyclooctyne |
| | | | containing | or |
| | | | phosphine | cyclooctyne |
| | | | (Staudinger | derivative |
| | | | reagent) | (SPAAC |
| | | | | reaction |
| | | | | reagent) |
| amine | activated ester | | epoxy | |
| norbornene | azide | | tetrazine | |
| tetrazine | some cyclooctynes or cyclooctyne | | norbornene | |
| | derivatives (e.g., BCN or trans- | | | |
| | cyclooctene (TCO)) | | | |
| sulfonyl fluoride | aryl fluorosulfate | | | |
| (SuFEx reagent) | | | | |
| epoxy | amine | | | |
| phenyl-containing | azide | | | |
| phosphine | | | | |
| (Staudinger | | | | |
| reagent) | | | | |
| acrylate | thiol | | | |
| alkyne | azide | | thiol | |
| cyclooctyne or | azide | tetrazine | thiol | |
| cyclooctyne | | | | |
| derivative | | | | |
| (SPAAC reagent) | | | | |
| some | azide | tetrazine | thiol | |
| cyclooctenes or | | | | |
| cyclooctene | | | | |
| derivatives | | | | |

| | |
|---|---|
| activated ester | amine |
| aryl fluorosulfate | sulfonyl fluoride (SuFEx reagent) |

In some instances, the substrate surface (e.g., the resin layer 18 or the single-layer version of the substrate 16) inherently includes functional groups that attach the functionalized focal point of attachment 28 to the surface. In other instances, the substrate surface (e.g., the resin layer 18 or the single-layer version of the substrate 16) is functionalized to add the functional groups that attach the functionalized focal point of attachment 28 to the surface. In one example, the substrate surface (e.g., the resin layer 18 or the single-layer version of the substrate 16) includes a silane that attaches the functionalized focal point of attachment 28 to the surface. The silane may be introduced to the substrate surface via a silanization process, which is described in more detail herein. In other examples, the substrate surface is free of silane.

In some instances, the substrate surface (e.g., the resin layer 18 or the polymeric hydrogel 24) of the flow cell 10 includes a regenerating moiety 42 (shown, in phantom, attached to one of the dendrons 26 in FIG. 1B), and the functionalized focal point of attachment 28 is attached to the substrate surface via bonding with the regenerating moiety 42. The regenerating moiety 42 may utilize covalent bonding, or non-covalent bonding. One example of the regenerating moiety 42 that uses covalent surface attachment includes 1,2,4-triazoline-3,5-dione (TAD). Another example of the regenerating moiety 42 that uses covalent surface attachment includes tetrazine attached to an amine of the polymeric hydrogel 24. In this example, the dendron 26 may include a bicyclononyne focal point 28. One example of the regenerating moiety 42 that uses non-covalent surface attachment includes alkyne-PEG4-biotin bonded to the substrate surface through the alkyne. In this example, streptavidin is introduced, either as part of the initial regenerating moiety 42, as part of the initial dendron 26, or separately from both the regenerating moiety 42 and the dendron 26, in order to link the biotin end of moiety 42 to a biotinylated focal point 28 of the dendron 26. Use of the regenerating moiety 42 is described in further detail in regard to the methods described hereinbelow.

As described, the focal point 28 of the dendron 26 is selected to be orthogonal to the plurality of peripheral functional groups 30 included in the dendron 26. Several suitable examples of orthogonal functionalities (e.g., for the focal point 28 and for the peripheral groups 30) are set forth in Table 2 below. For each of the given focal points 28, any of the listed peripheral groups 30 may be used. It is to be understood that when multiple rows are associated with a given focal point 28, any one of the listed peripheral groups 30 may be used.

The following key is used in Table 2: Azide=AZ; Amine=AM; Tetrazine=T; Sulfonyl fluoride (SuFEx reagent)=SuFEx; Thiol=SH; Epoxy=E; Activated Ester=AE; Aryl fluorosulfate=AF; Phenyl-containing phosphine (Staudinger reagent)=PCP; Alkyne=AL; cyclooctyne or cyclooctyne derivative (SPAAC reagent)=CO; Norbornene=N; Acrylate=AC; and Terminal Alkene=TA.

TABLE 2

| Dendron | | | | | | | | | |
|---|---|---|---|---|---|---|---|---|---|
| Focal | Corresponding Orthogonal | | | | | | | | |
| Point | Peripheral Functional Group | | | | | | | | |
| AZ | AM | T | SuFEx | SH | E | AE | AF | | |
| AM | AZ | T | SuFEx | SH | PCP | AL | CO | AF | |
| N | SuFEx | E | PCP | AC | AL | CO | AE | AF | |
| T | AZ | AM | SuFEx | SH | AL | PCP | AC | AE | AF |
| SuFEx | AZ | AM | N | T | SH | PCP | CO | AE | AC |
| | E | | | AF | | TA | | AL | |
| SH | AZ | AM | T | SuFEx | PCP | | | AF | |
| E | AZ | N | T | SuFEx | PCP | AC | AL | CO | |
| | TA | | AE | | | AF | | | |
| PCP | AL | N | T | SuFEx | SH | E | AC | AM | |
| | TA | CO | | AE | | AF | | | |
| AC | AE | T | SuFEx | E | PCP | CO | AL | N | AF |
| AL | AM | N | SuFEx | E | PCP | AC | CO | TA | AE |

| | AF | | | | | | | |
|---|---|---|---|---|---|---|---|---|
| CC | AM | N | SuFEx | E | PCP | AC | | AL |
| AE | AZ | N | SuFEx | E | PCP | AC | AL | T |
| AF | AZ | AM | SH | T | N | E | PCP | AC |

| | |
|---|---|
| AL | SuFEx |

The chemical functionality of each of the peripheral groups 30 is also selected to be compatible (i.e., capable of reactive bonding) with primers included in a primer set 32. Several suitable examples of compatible functionalities (e.g., for the peripheral groups 30 and for 5′ end of the primers) are set forth in Table 3. For each of the given peripheral groups 30, any of the listed 5′ end groups may be used.

TABLE 3

| Peripheral Functional Group(s) | 5′ End of Primer | | | | | |
|---|---|---|---|---|---|---|
| azide | alkyne | norbornene | | phenyl- | | cyclooctyne |
| | | | | containing | | or |
| | | | | phosphine | | cyclooctyne |
| | | | | (Staudinger | | derivative |
| | | | | reagent) | | (SPAAC |
| | | | | | | reaction |
| | | | | | | reagent) |
| amine | activated ester | | | epoxy | | |
| norbornene | azide | | | tetrazine | | |
| tetrazine | cyclooctyne or | | | norbornene | | |
| | cyclooctyne derivative | | | | | |
| | (SPAAC reaction | | | | | |
| | reagent) | | | | | |
| sulfonyl fluoride (SuFEx reagent) | aryl fluorosulfate | | | | | |
| epoxy | amine | | | | | |
| phenyl-containing phosphine | azide | | | | | |
| (Staudinger reagent) | | | | | | |
| acrylate | thiol | | | | | |
| alkyne | azide | | | thiol | | |
| cyclooctyne or cyclooctyne | azide | | tetrazine | | thiol | |
| derivative (SPAAC reagent) | | | | | | |
| some cyclooctenes or cyclooctene | azide | | tetrazine | | thiol | |
| derivatives | | | | | | |

| | |
|---|---|
| activated ester | amine |
| aryl fluorosulfate | sulfonyl fluoride (SuFEx reagent) |

Further specific examples of compatible functional groups for (i) 5′ ends of the primers and (ii) peripheral groups 30 are described hereinbelow.

In some examples, the functionality chosen for 5′ end of the primers may be orthogonal to the functionality of the resin layer 18 or the polymeric hydrogel 24 to which the focal point 28 of the dendron architecture 26 is attached. Such functionalities may prevent primers from grafting to the polymeric hydrogel 24 or the resin layer 18 (rather than grafting to the peripheral groups 30 of the dendron architecture 26) and mitigate interference from such primers during sequencing operations.

One specific example of a suitable dendron architecture 26 is represented by formula (I):

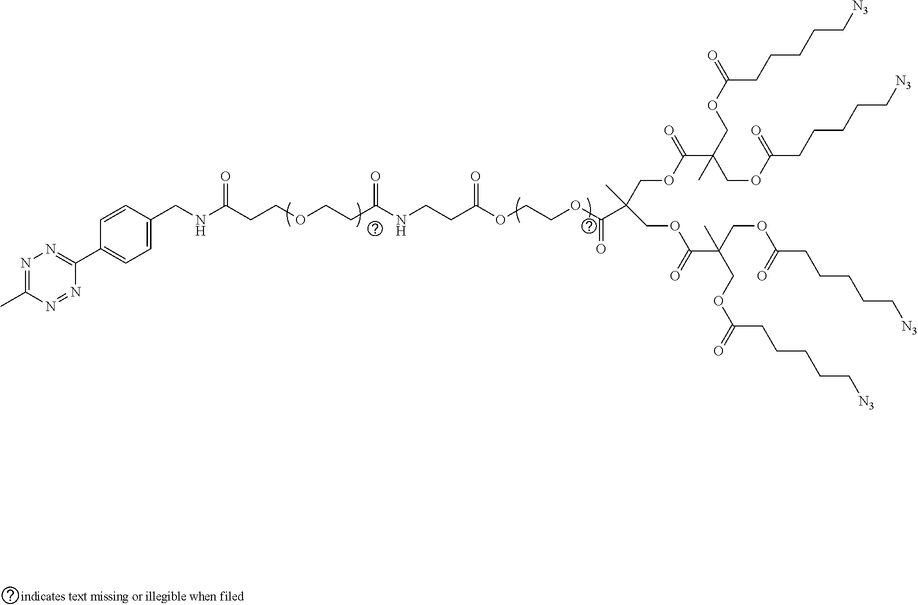

The dendron architecture 26 represented by formula (I) includes tetrazine as the focal point 28 and azides as the peripheral groups 30. The azides are used to attach the primers of the primer set 32, and the tetrazine is used to attach the dendron architecture 26 to the resin layer 18 or the polymeric hydrogel 24. While the structure depicted in formula (I) includes the functional groups shown, it is to be understood that other functional groups may be utilized in lieu of the azides and/or the tetrazine, such as those set forth in Table 1 and Table 2 herein. It is believed that the tetrazine (as the focal point 28) and azides (as the peripheral groups 30) in formula (I) may be replaced with other orthogonal focal points 28 and peripheral groups 30 as described herein.

As shown in one of the depressions 20 of FIG. 1D, the dendron architecture 26 includes primers (included as part of the primer set 32) grafted to the peripheral groups 30. The primer set 32 includes two different primers that may be used in sequential paired end sequencing. As examples, the primer set 32 may include P5 and P7 primers, P15 and P7 primers, or any combination of the PA primers, the PB primers, the PC primers, and the PD primers set forth herein. As further examples, the primer set 32 may include any two PA, PB, PC, and PD primers, or any combination of one PA primer and one PB, PC, or PD primer, or any combination of one PB primer and one PC or PD primer, or any combination of one PC primer and one PD primer. Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYER™, and other instrument platforms. The P5 and P7 primers have a universal sequence for seeding and/or amplification purposes.

The P5 primer which is a cleavable primer due to the cleavable nucleobase uracil or “n”) is:

P5 #1: 5′ → 3′
(SEQ. ID. NO. 1)
AATGATACGGCGACCACCGAGAUCTACAC
P5 #2: 5′ → 3′
(SEQ. ID. NO. 2)
AATGATACGGCGACCACCGAGAnCTACAC

where “n” is inosine in SEQ. ID. NO. 2; or

P5 #3: 5′ → 3′
(SEQ. ID. NO. 3)
AATGATACGGCGACCACCGAGAnCTACAC

where “n” is alkene-thymidine (i.e., alkene-dT) in SEQ. ID. NO. 3.

The P7 primer (also cleavable) may be any of the following:

P7 #1: 5′ → 3′
(SEQ. ID. NO. 4)
CAAGCAGAAGACGGCATACGAnAT
P7 #2: 5′ → 3′
(SEQ. ID. NO. 5)
CAAGCAGAAGACGGCATACnAGAT
P7 #3: 5′ → 3′
(SEQ. ID. NO. 6)
CAAGCAGAAGACGGCATACnAnAT

where “n” is 8-oxoguanine in each of the sequences.

The P15 primer is:

P15: 5′ → 3′
(SEQ. ID. NO. 7)
AATGATACGGCGACCACCGAGAnCTACAC

where “n” is allyl-T (a thymine nucleotide analog having an allyl functionality).

The other primers (PA-PD) mentioned above include:

PA 5′ → 3′
(SEQ. ID. NO. 8)
GCTGGCACGTCCGAACGCTTCGTTAATCCGTTGAG
PB 5′ → 3′
(SEQ. ID. NO. 9)
CGTCGTCTGCCATGGCGCTTCGGTGGATATGAACT
PC 5′ → 3′
(SEQ. ID. NO. 10)
ACGGCCGCTAATATCAACGCGTCGAATCCGCAACT
PD 5′ → 3′
(SEQ. ID. NO. 11)
GCCGCGTTACGTTAGCCGGACTATTCGATGCAGC

While not shown in the example sequences for PA-PD, it is to be understood that any of these primers may include a cleavage site, such as uracil, 8-oxoguanine, allyl-T, etc. at any point in the strand.

It is to be understood that the cleavage sites of the primers in the primer set 32 are orthogonal to each other (i.e., one cleavage site is not susceptible to the cleaving agent used for the other cleavage site), so that after amplification, forward or reverse strands can be cleaved, leaving the other of the reverse or forward strands for sequencing.

The 5′ terminal end of each primer includes a functional group that can attach to the peripheral groups 30 of the dendron architecture 26. The functional group at 5′ terminal end of the primers enables the immobilization of the primers by single point covalent attachment. The attachment will depend, in part, on the functional groups of the peripheral groups 30. Examples of terminated primers that may be used include an alkyne terminated primer (e.g., including a 5′ hexynyl group), a tetrazine terminated primer, an azido terminated primer, an amino terminated primer, an epoxy or glycidyl terminated primer, a thiophosphate terminated primer, a thiol terminated primer, an aldehyde terminated primer, a hydrazine terminated primer, and a triazolinedione terminated primer.

In some specific examples, an amine terminated primer may be reacted with succinimidyl (NHS) peripheral groups 30, an aldehyde terminated primer may be reacted with hydrazine peripheral groups 30, an alkyne terminated primer may be reacted with an azide peripheral groups 30, an azide terminated primer may be reacted with an alkyne or DBCO (dibenzocyclooctyne) peripheral groups 30, an amino terminated primer may be reacted with an activated carboxylate peripheral groups 30 or with NHS ester peripheral groups 30, a thiol terminated primer may be reacted with an alkylating reactant (e.g., iodoacetamine or maleimide) in the peripheral groups 30 or with a tetrazole of the peripheral groups 30, or a phosphoramidite terminated primer may be reacted with a thioether peripheral groups 30.

While several examples have been provided, it is to be understood that any pair of compatible functional groups depicted in Table 3 hereinabove may be used for the peripheral groups 30 and for 5′ end of the primers.

The primers may also include a linker between the primer sequence and the 5′ terminal end group. Example linkers include a polyT sequence. In some examples, the polyT region includes from 2 T bases to 20 T bases. As specific examples, the polyT region may include 3, 4, 5, 6, 7, or 10 T bases. Other suitable linkers are non-nucleic acid linkers, such as a polyethylene glycol chain, an alkyl group or a carbon chain, an aliphatic linker with vicinal diols, a peptide linker, etc. The following are some examples of nucleotides including non-nucleic acid linkers with terminal alkyne groups (where B is the nucleobase and “oligo” is the primer sequence):

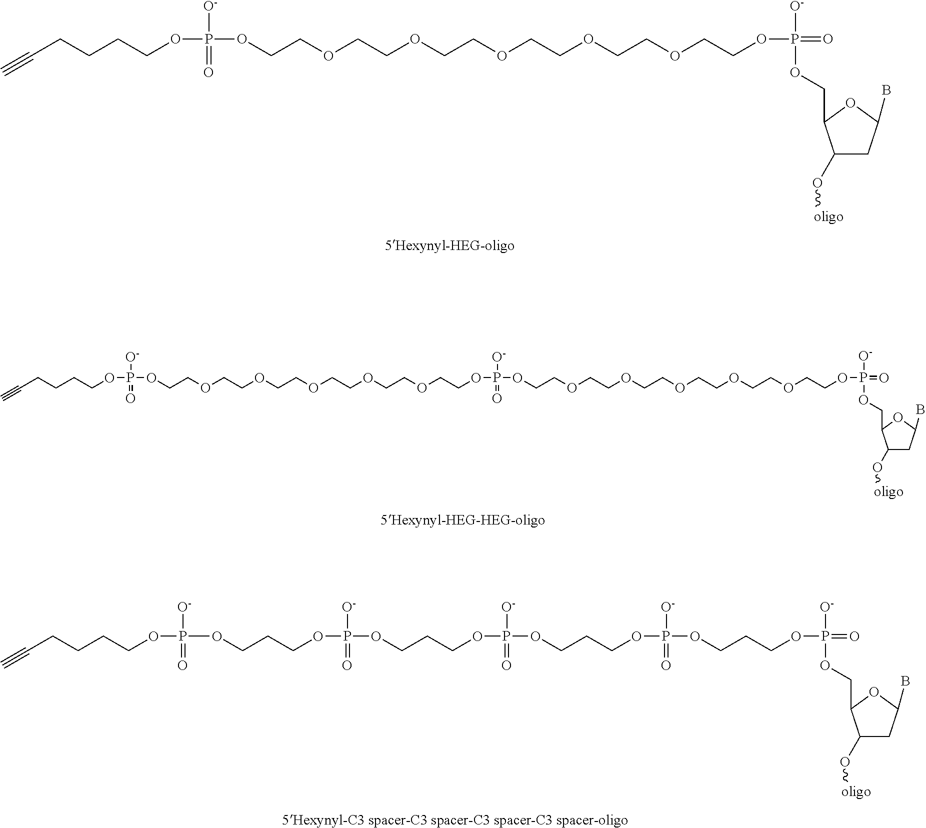

Several methods of forming patterned flow cells 10 that include the dendron architecture 26 will now be described.

### Method of Forming a Hydrogel-Inclusive Flow Cell

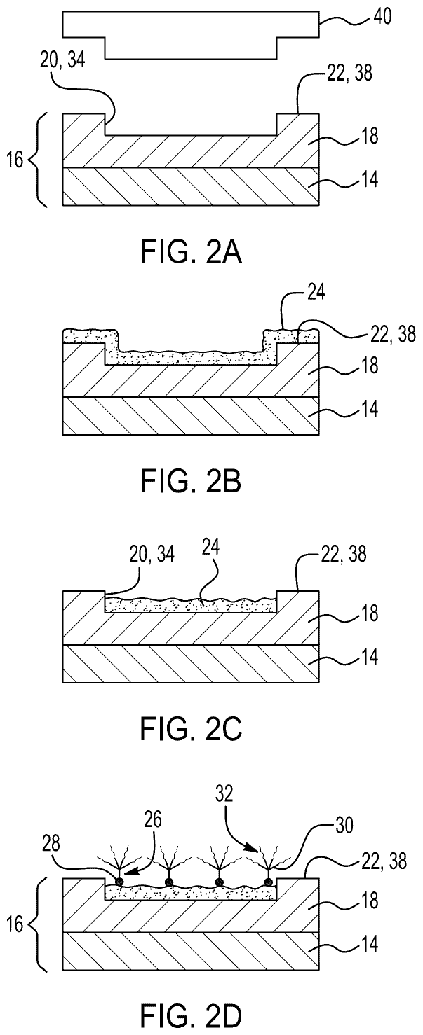

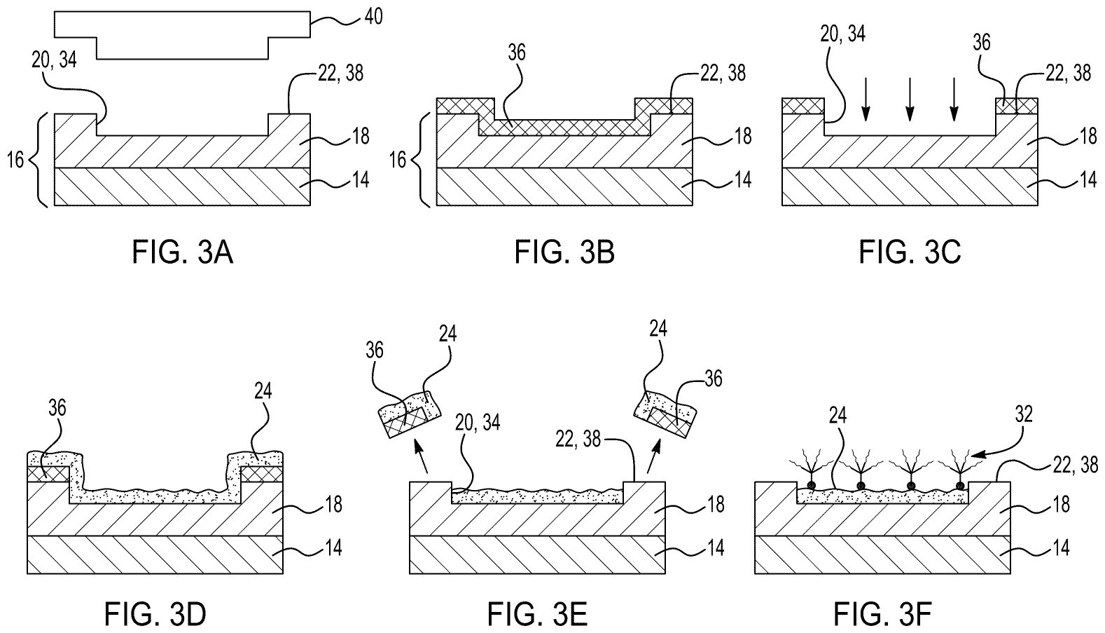

Example methods that utilize the hydrogel 24 in the flow cell 10 are depicted in FIG. 2A through FIG. 2D and in FIG. 3A through FIG. 3F.

The method depicted in FIG. 2A through FIG. 2D generally includes depositing a polymeric hydrogel 24 over a surface of a substrate 16; and introducing a dendron architecture 26 to a surface of the polymeric hydrogel 24, wherein the dendron architecture 26 includes: a functionalized focal point of attachment 28 that attaches to the polymeric hydrogel 24 surface, a plurality of peripheral functional groups 30 that are orthogonal to the functionalized focal point of attachment 28, and a primer set 32 grafted to the plurality of peripheral functional groups 30 prior to the introduction of the dendron architecture 26 to the polymeric hydrogel 24 surface.

It is to be understood that the examples of the method shown in FIG. 2A through FIG. 2D may be performed using a single-layer version of the substrate 16 including the base support 14 (without layer 18), or with a multi-layer version of the substrate 16 including the resin layer 18 applied over the base support 14, as shown. As such, each of the method steps described below in regard to the resin layer 18 of the multi-layer version of the substrate 16 is also applicable to a single-layer substrate.

Any suitable materials described herein may be used for the resin layer 18 and/or the base support 14 of the substrate 16. Further, any suitable deposition technique disclosed herein may be used to deposit the resin 18 (that forms the resin layer 18) on the base support 14 of the substrate 16.

After the resin layer 18 has been applied to the base support 14, the lane 34 and the bonding region 38 or the depression 20 and interstitial regions 22 are defined in the resin layer 18. While a single depression 20 is shown in the method depicted in FIG. 2A through FIG. 2D, it is to be understood that the substrate 16 may include the lane 34, or a plurality of depressions 20, where each individual depression 20 is separated from each other depression 20 by interstitial regions 22 (similar to the example shown in FIG. 1C).

The lane 34 or depression(s) 20 may be formed in the layer 18 using any suitable technique described herein, such as nanoimprint lithography (NIL) or photolithography, etc. As one example of forming the lane 34 or depression 20 in the layer 18, a working stamp 40 including feature(s), which is/are a negative replica of the lane 34 or the depression(s) 20, may be used. In this example, the working stamp 40 is pressed into the resin layer 18 while the resin layer 18 is soft, which creates an imprint of the feature(s) of the working stamp 40 in the layer 18. The resin layer 18 may then be cured with the working stamp 40 in place. Curing may be accomplished by exposure to actinic radiation or heat. In an example, curing of the resin may be accomplished by exposing the applied resin to incident light at an energy dose ranging from about 0.5 J to about 20 J for 30 seconds or less. The incident light may be actinic radiation, such as ultraviolet (UV) radiation. The curing process may include a single UV exposure stage.

After the material for the resin layer 18 is applied to the base support 14, the material may be soft baked to remove any excess solvent that is present in the material. The soft bake may take place at a lower temperature than is used for curing the material (e.g., ranging from about 50° C. to about 150° C.) and for a time ranging from greater than 0 seconds to about 3 minutes. In an example, the soft bake time ranges from about 30 seconds to about 2.5 minutes.

After curing, and in some instances the post-curing bake, the working stamp 40 is released and the resin layer 18 is formed. As shown in FIG. 2A, the release of the working stamp 40 from the resin layer 18 creates the depression(s) 20 (or the lane 34 in other examples) in the layer 18.

With the depression 20 formed in the resin layer 18, an example of the method continues with the application of the polymeric hydrogel 24 over the resin layer 18 (e.g., over the depression 20 and over the interstitial regions 22). This is shown in FIG. 2B. The polymeric hydrogel 24 may be any of the hydrogel materials described herein and may be deposited using any suitable technique described herein.

While not shown in the figures, in some instances, the method further includes silanizing the resin layer 18 prior to depositing the polymeric hydrogel 24 thereon. Silanization (or activation) of the resin layer 18 may be accomplished by exposure of the resin layer 18 to a silanizing solution (e.g., trimethoxysilane in an organic solvent, such as an alcohol, a hydrocarbon, or acetone). Silanization is one example of a process that can be used to introduce functional groups to the resin layer 18 that can attach the polymeric hydrogel 24. Another example of a suitable activation process is plasma ashing of the resin layer 18, which introduces hydroxyl groups.

In some instances, prior to being deposited in the depression(s) 20 or lane 34, the polymeric hydrogel 24 may first be diluted using water or another suitable solvent (e.g., up to 10% dilution). Examples of suitable solvents for the hydrogel 24 include dimethyl sulfoxide (DMSO), isopropyl alcohol (IPA), or a mixture of either of these solvent with water.

In some instances, following the deposition of the polymeric hydrogel 24, the hydrogel 24 may then be exposed to UV light (e.g., for polymerization, curing, drying).

As shown in FIG. 2C, following the application of the polymeric hydrogel 24 in the depression(s) 20 and over the interstitial regions 22 (or lane 34 and bonding regions 38), the method continues by polishing the polymeric hydrogel 24 from the interstitial regions 22 (and/or the bonding region 38). The polishing process removes the polymeric hydrogel 24 from the interstitial regions 22 (and/or the bonding region 38), while leaving the polymeric hydrogel 24 within the depression(s) 20 (or lane 34) intact. Polishing of the polymeric hydrogel 24 may be accomplished using a chemical slurry (including, e.g., an abrasive, a buffer, a chelating agent, a surfactant, and/or a dispersant). Alternatively, polishing may be performed with a solution that does not include abrasive particles. The chemical slurry may be used in a chemical mechanical polishing system to polish the interstitial regions 22 (and/or the bonding region 38). Polishing head(s)/pad(s) or other polishing tool(s) may be used that is/are capable of polishing the polymeric hydrogel 24 that is present over the interstitial regions 22 (and/or the bonding region 38), while leaving the polymeric hydrogel 24 in the depression(s) 20 (or lane 34) at least substantially intact. As an example, the polishing head may be a Strasbaugh ViPRR II polishing head. The polishing process can remove the polymeric hydrogel 24 from the interstitial regions 22 (and/or the bonding region 38) without deleteriously affecting the underlying resin layer 18.

Cleaning and drying processes may be performed after polishing. The cleaning process may utilize a water bath and sonication. The water bath may be maintained at a relatively low temperature ranging from about 22° C. to about 30° C. The drying process may involve spin drying, or drying via another suitable technique.

After the polymeric hydrogel 24 has been polished from the interstitial regions 22 (and/or the bonding region 38), this example method proceeds by introducing the dendron 26 to the polymeric hydrogel 24 in the depression(s) 20 or lane 34. This is shown in FIG. 2D. As shown in the figure, the dendron 26 includes primers of the primer set 32, where the primers have been grafted to the plurality of peripheral functional groups 30 prior to the introduction of the dendron 26 to the polymeric hydrogel 24 surface. Pre-grafting the primers to the peripheral functional groups 30 (prior to introducing the dendron 26 to the polymeric hydrogel 24) may enhance signal strength during sequencing operations and/or simplify overall workflows.

Introduction of the dendron 26 to the polymeric hydrogel 24 may be accomplished using any suitable technique, such as spin coating, dunk coating, puddle dispensing, or a flow-through technique (e.g., after bonding of two substrates 16 or a substrate 16 and a lid or using a temporary lid). When the dendron architecture 26 comes into contact with the polymeric hydrogel 24, the focal point 28 interacts (e.g., forms a chemical bond) with functional groups of the polymeric hydrogel 24, thereby forming an attachment point and anchoring the dendron 26 to the polymeric hydrogel 24 via the focal point 28.

While not shown in FIG. 2A through FIG. 2D, in some instances, the dendron architecture 26 may become attached to the polymeric hydrogel 24 through an intervening regenerating moiety 42 (see FIG. 1D). In these examples, prior to introducing the dendron architecture 26 to the surface of the polymeric hydrogel 24, the method further comprises grafting the regenerating moiety 42 to the surface of the polymeric hydrogel 24. Further in these examples, the focal point 28 attaches to the regenerating moiety 42 (e.g., instead of the focal point 28 attaching directly to the polymeric hydrogel 24). Examples of suitable regenerating moieties 42 include those described hereinabove. As an example, when the regenerating moiety 42 is alkyne-PEG4-biotin, the biotin is bonded to streptavidin, which is also bonded to a second biotin that is the focal point 28 of the dendron 16. In this example, the alkyne-PEG4-biotin bonds to the polymeric hydrogel 24 through the alkyne, and the biotin focal point 28 of the dendron 26 bonds to the biotin of the alkyne-PEG4-biotin through the streptavidin. Following sequencing operations (e.g., clustering and sequencing of target analytes), a reagent may be introduced to the substrate surface to break the biotin-streptavidin bonds, which removes the biotinylated dendrons 26 (and the primers attached thereto) and also re-exposes the biotin of the regenerating moiety 42. A suitable reagent for the alkyne-PEG4-biotin/streptavidin/biotin regenerating moiety is hot formamide.

While the dendron architecture 26 depicted in FIG. 2D includes four visible peripheral groups 30, it is to be understood that other amounts of peripheral groups 30 may be included in the dendron 26. In an example, the number of peripheral groups 30 included in the dendron architecture ranges from 2 to 20.

As described herein in regard to Table 2, the focal point 28 of the dendron 26 is selected to be orthogonal to the peripheral groups 30. The focal point 28 and the peripheral groups 30 may include any of the example functional groups set forth herein. In one specific example, the functionalized focal point of attachment 28 is an N-hydroxysuccinimide, the plurality of peripheral groups 30 includes a plurality of azides, and the polymeric hydrogel surface includes an amine functional group that attaches the functionalized focal point of attachment 28 to the polymeric hydrogel surface.

As mentioned, the primers in this example of the method are pre-grafted to the peripheral groups 30 of the dendron 26. Pre-grafting of the primers to the peripheral groups 30 (prior to introducing the dendron 26 to the hydrogel 24) may be accomplished using any suitable grafting technique. As an example, pre-grafting of the primers to the peripheral groups 30 may be accomplished using an incubation process, during which the coupling reaction takes place. This example of grafting may utilize a solution or mixture, which includes the dendron architecture 26, the primers of the primer set 32, water, a buffer, and a catalyst. It is to be understood that the buffer and the catalyst, as well as other additives, may or may not be included in the primer solution or mixture during the incubation process. Regardless of the grafting method used, the primers of the primer set 32 attach to the reactive peripheral groups 30 of the dendron 26, and have no affinity for the focal point 28, due in part to the orthogonal chemistries of the peripheral groups 30 and the focal point 28.

The patterned structure that is formed and that is shown in FIG. 2D can then be used in an open wafer sequencing process, or may be bonded to the lid or to another patterned structure using the spacer layer and a suitable bonding method. The lid and the patterned structure or the two patterned structures may be bonded using any suitable technique, such as laser bonding, diffusion bonding, anodic bonding, eutectic bonding, plasma activation bonding, glass frit bonding, or others methods known in the art.

Referring now to FIG. 3A through FIG. 3F, the method depicted here is similar to the method depicted in FIG. 2A through FIG. 2D, except that a sacrificial layer 36 is used to selectively deposit the polymeric hydrogel 24 in the depression(s) 20 (or lane 34) (such that the interstitial regions 22 and/or bonding region 38) remain free of polymeric hydrogel 24). As such, in the examples depicted in FIG. 3A through FIG. 3F, a polishing process is not used to remove the polymeric hydrogel 24 from the interstitial regions 22.

Similar to the method shown in FIG. 2A through FIG. 2D, it is to be understood that the examples of the method shown in FIG. 3A through FIG. 3F may be performed with or without the base support 14 (e.g., upon which the resin material of the resin layer 18 may be applied and cured). As such, the processes described below are also applicable to a single-layer version of the substrate 16, which includes the base support 14 without the resin layer 18 applied thereon.

Any suitable resin material described herein may be used to form the resin layer 18. Further, any suitable deposition technique disclosed herein may be used to deposit the resin material (that forms the resin layer 18) on the base support 14. Still further, the base support 14 may be any of the base support materials described herein.

After the resin layer 18 has been applied to the base support 14, the depression(s) 20 and interstitial regions 22 or the lane 34 and bonding region 38 are defined in the resin layer 18 as described in reference to the FIG. 2 series. While a single depression 20 or lane 34 is shown in the method depicted in FIG. 3A through FIG. 3F, it is to be understood that the flow cell 10 may include a plurality of depressions 20, where each individual depression 20 is separated from each other depression 20 by interstitial regions 22 (similar to that shown in FIG. 1C and FIG. 1D). FIG. 3A specifically illustrates the release of the working stamp 40 and the formation of the resin layer 18. In this particular example, the release of the working stamp 40 from the resin layer 18 creates the depression 20 or lane 34 in the layer 18.

With the depression(s) 20 or lane 34 formed in the resin layer 18, this example method continues with the application of the sacrificial layer 36 over the resin layer 18 (e.g., over the depression(s) 20 or lane 34 and over the interstitial regions 22 and/or bonding region 38), as shown in FIG. 3B. Examples of suitable materials for the sacrificial layer 36 include metals (e.g., aluminum, copper, titanium, gold, silver, etc.), photoresists, and nitrides (silicon, aluminum, tantalum, etc.). Further examples of the sacrificial layer 36 include semi-metals, such as silicon and germanium. In some examples, the semi-metal or metal may be at least substantially pure (<99% pure). In other examples, molecules or compounds of the listed elements may be used, as long as they provide the desired etch stop or other function in a particular method. For example, oxides of any of the listed semi-metals (e.g., silicon dioxide) or metals (e.g., aluminum oxide) may be used, alone or in combination with the listed semi-metal or metal. As another example, silicon nitride may be used, either alone or in combination with silicon. As further examples, aluminum nitride may be used (either alone or in combination with aluminum), or tantalum nitride may be used (either alone or in combination with tantalum). The deposition technique used for the sacrificial layer 36 may depend, in part, upon the material used for the sacrificial layer 36 and upon the material(s) used for the substrate 16.

In some examples, selective deposition techniques (such as chemical vapor deposition (CVD) and variations thereof (e.g., low-pressure CVD (LPCVD)), atomic layer deposition (ALD), and/or masking techniques) may be used to apply the sacrificial layer 36 at desirable areas of the substrate 16. One of these examples involves: applying the sacrificial layer 36 over the depression(s) 20 and the interstitial regions 22 or the lane 34 and the bonding region 38, and etching the sacrificial layer 36 from within the depression(s) 20 or lane 34, whereby the sacrificial layer 36 remains on the interstitial regions 22 and/or the bonding region 38. This example is depicted in FIG. 3B and FIG. 3C, and will now be described.

As shown in FIG. 3B, the sacrificial layer 36 is applied within the depression(s) 20 or lane 34 and over the interstitial regions 22 and/or the bonding region 38. The sacrificial layer 36 may be applied to cover a bottom surface of the depression(s) 20 or lane 34 and to cover the interstitial regions 22 and/or the bonding region 38 (as shown in FIG. 3B). The sacrificial layer 36 may conformally coat the layer 18 (and thus covers at least some sidewalls of the depression(s) 20 or lane 34, or so that the depression(s) 20 or lane 34 is filled with the sacrificial layer 36.

As shown in FIG. 3C, portions of the sacrificial layer 36 within the depression(s) 20 or lane 34 (and not over the interstitial regions 22 and/or bonding region 38) may then be etched (as shown by the arrows in FIG. 3C). Any suitable etching technique may be used that can selectively remove portions of the sacrificial layer 36 from within the depression(s) 20 or lane 34, while leaving portions of the sacrificial layer 36 on the interstitial regions 22 and/or the bonding region 38 intact. The etching technique used may depend, in part, upon the material used for the sacrificial layer 36. Etching may be performed using a dry etch process, or a wet etch process. As examples, aluminum sacrificial layer can be removed in acidic or basic conditions, a copper sacrificial layer can be removed using FeCl₃, a copper, gold or silver sacrificial layer can be removed in an iodine and iodide solution, a titanium sacrificial layer can be removed using H₂O₂, a silicon sacrificial layer can be removed in basic (pH) conditions, a silicon dioxide sacrificial layer can be removed using a hydrofluoric acid (HF) etch, and silicon nitride sacrificial layer can be removed using a phosphoric acid etch. As further examples, a reactive ion etch (e.g., with 10% CF₄ and 90% O₂) may be used that etches the sacrificial layer 36 at a rate of about 17 nm/min. In another example, a 100% O₂ plasma etch may be used that etches the sacrificial layer 36 at a rate of about 98 nm/min. Other suitable sacrificial layer 36 etchants include CF₄/O₂/N₂, CHF₃/O₂, and CHF₃/CO₂. As still other specific examples, a CHF₃ and O₂ and Ar reactive ion etch may be used for a silicon dioxide sacrificial layer 36 or SF₆ and O₂ or CF₄ and O₂ or CF₄ may be used for a silicon nitride sacrificial layer 36.

The resin layer 18 may function as an etch stop to sacrificial layer 36 etching, e.g., when the layer 18 has a slower etch rate than the sacrificial layer 36.

In examples in which the sacrificial layer 36 is a photoresist, the photoresist may be any suitable negative or positive photoresist. In these examples, the sacrificial layer 36 may be exposed to light so that an insoluble portion of the photoresist is formed over interstitial regions 22 and/or bonding region 38 and a soluble portion of the photoresist is formed (and subsequently removed) from within the depression(s) 20 or lane 34. This creates a mask over the interstitial regions 22 and/or bonding region 38.

Examples of suitable negative photoresists that may be used as the sacrificial layer 36 include those in the NR® series of photoresists (available from Futurrex), or in the SU-8 Series of photoresists, or in the KMPR® Series of photoresists (the two latter of which are available from Kayaku Advanced Materials, Inc.), or in the UVN™ Series of photoresists (available from DuPont). When the negative photoresist sacrificial layer 36 is used, it is selectively exposed to certain wavelengths of light to form an insoluble negative photoresist over the interstitial regions 22 and/or the bonding region 38, and is exposed to a developer to remove soluble portions (e.g., those portions that are not exposed to the certain wavelengths of light) from the depression(s) 20 or lane 34.

Examples of suitable positive photoresists that may be used as the sacrificial layer 36 include those in the MICROPOSIT® S1800 series or the AZ® 1500 series, both of which are available from Kayaku Advanced Materials, Inc. Another example of a suitable positive photoresist is SPR™-220 (from DuPont). When a positive photoresist is used, selective exposure to certain wavelengths of light forms a soluble region (e.g., which is at least 95% soluble in a developer) in the depression(s) 20 or lane 34, and the developer is used to remove the soluble regions. Those portions of the positive photoresist overlying the interstitial regions 22 and/or bonding region 38 are not exposed to light, and will become insoluble in the developer. The insoluble positive photoresist thus remains (and forms a mask) over the interstitial regions 22 and/or the bonding region 38.

The soluble portions are removed with a suitable developer so that the surface of the substrate 16 that forms the bottom of the depression(s) 20 or lane 34 is exposed. Examples of suitable developers for the negative photoresist include aqueous-alkaline solutions, such as diluted sodium hydroxide, diluted potassium hydroxide, or an aqueous solution of the metal ion free organic TMAH (tetramethylammonium hydroxide). Examples of suitable developers for the positive photoresist include aqueous-alkaline solutions, such as diluted sodium hydroxide, diluted potassium hydroxide, or an aqueous solution of the metal ion free organic TMAH (tetramethylammonium hydroxide).

As shown in FIG. 3C, removal of the sacrificial layer 36 from within the depression(s) 20 or lane 34 exposes a surface of the resin layer 18 that makes up the bottom of the depression(s) 20 or lane 34, where the polymeric hydrogel 24 is to be applied. However, as further shown in FIG. 3C, the sacrificial layer 36 remains on the interstitial regions 22 and/or the bonding region 38.

As shown in FIG. 3D, the polymeric hydrogel 24 may then be applied within the depression(s) 20 or lane 34 and over the (remaining) sacrificial layer 36 overlying the interstitial regions 22 and/or the bonding region 38. The polymeric hydrogel 24 may be any of the hydrogel materials described herein and may be deposited using any suitable technique described herein. In some instances, the method further includes activating the resin layer 18 within the depression(s) 20 or lane 34 prior to depositing the polymeric hydrogel 24 thereon. Activation of the resin layer 18 may be accomplished by exposure of the resin layer 18 to a suitable silanizing solution or through plasma ashing of the resin layer 18. The polymeric hydrogel 24 may be exposed to UV light to initiate polymerization, curing, drying, etc.

As shown in FIG. 3E, following the application of the polymeric hydrogel 24 in the depression(s) 20 or lane 34 and over the remaining material of the sacrificial layer 36, the method continues by removing the remaining sacrificial layer 36 (and the polymeric hydrogel 24 applied thereon) from the interstitial regions 22 and/or the bonding region 38. Removal of the sacrificial layer 36 may be accomplished using a suitable technique, such as a lift-off process. The lift-off process may involve an organic solvent that is capable of dissolving or otherwise lifting off the sacrificial layer 36, without deleteriously affecting the polymeric hydrogel 24 within the depression(s) 20 or lane 34. As examples, an aluminum sacrificial layer 36 (and the polymeric hydrogel 24 thereon) may be lifted-off using AZ® 400K (available from Microchemicals GmbH), and a silicon nitride sacrificial layer 36 (and the polymeric hydrogel 24 thereon) may be lifted-off using KOH, AZ® 400K, citric acid, tartaric acid, HELLMANEX® (an alkaline cleaning concentrate available from Hellma) and the like. The reagent used to lift-off the sacrificial layer 36 will depend upon the material used for the sacrificial layer 36. The material of the sacrificial layer 36 is soluble (at least 99% soluble) in the solvent used in the lift-off process. The lift-off process removes i) at least 99% of the sacrificial layer 36 material and ii) the polymeric hydrogel 24 positioned thereon. The lift-off process does not remove the portion of the polymeric hydrogel 24 that overlies (and is attached to) the layer 18 in the depression(s) 20 or lane 34 (due in part to the interaction with the activated resin layer 18).

After the remaining sacrificial layer 36 (and the polymeric hydrogel 24 thereon) has been removed from the interstitial regions 22 and/or the bonding region 38, this example method proceeds by introducing the dendron 26 to the polymeric hydrogel 24, such that the focal point 28 of the dendron 26 attaches to the polymeric hydrogel 24. This is depicted in FIG. 3F. As shown in the figure, the dendron 26 includes primers of a primer set 32 that have been pre-grafted to the peripheral groups 30 as described herein.

While not shown in FIG. 3A through FIG. 3F, in some instances, the regenerating moiety 42 is part of the mechanism that attaches the dendron architecture 26 to the polymeric hydrogel 24, as described hereinabove.

Introduction of the dendron 26 to the polymeric hydrogel 24 may be accomplished using any suitable technique as described herein in reference to FIG. 2D. When the dendron architecture 26 comes into contact with the polymeric hydrogel 24, the focal point 28 interacts (e.g., forms a chemical bond) with functional groups of the polymeric hydrogel 24, thereby forming an attachment point and directly or indirectly anchoring the dendron 26 to the polymeric hydrogel 24. While the dendron architecture 26 depicted in FIG. 3F includes four visible peripheral groups 30, each having a primer grafted thereto, it is to be understood that other amounts of peripheral groups 30 may be included in the dendron 26. In an example, the number of peripheral groups 30 included in the dendron 26 ranges from 2 to 20.

Any of the chemical functionalities set forth herein may be used for the focal point 28, for the peripheral groups 30, and for 5′ end of the primers of the primer set 32. As described in regard to Table 2, however, the focal point 28 of the dendron 26 may be selected to be orthogonal to the peripheral groups 30.

Pre-grafting of the primers to the peripheral groups 30 (prior to introducing the dendron 26 to the polymeric hydrogel 24) may be accomplished using any suitable method described herein. As an example, pre-grafting of the primers to the peripheral groups 30 may be accomplished using an incubation process as described herein. With any of the grafting methods, the primers of the primer set 32 attach to the reactive peripheral groups 30 of the dendron 26, and have no affinity for the any unreacted focal points 28.

The patterned structure that is formed and that is shown in FIG. 3F can then be used in an open wafer sequencing process, or may be bonded to the lid or to another patterned structure using the spacer layer and a suitable bonding method as described herein.

### Method of Forming a Hydrogel-Free Flow Cell

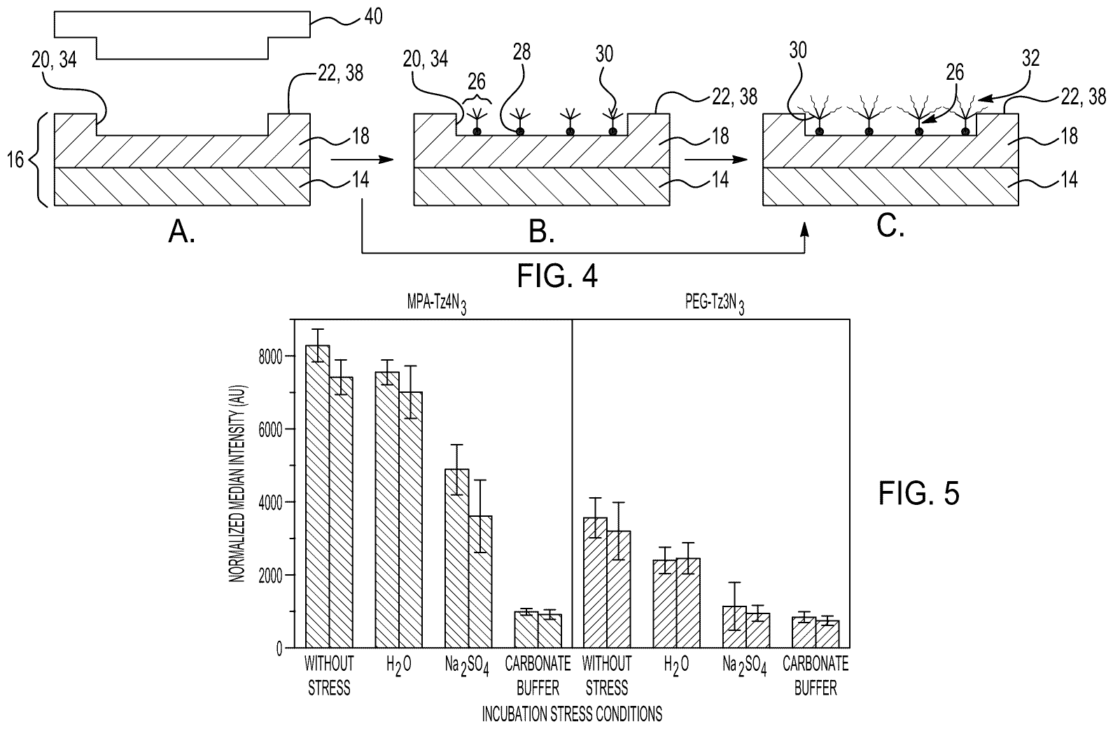

Two example methods are depicted in FIG. 4, one of which includes the processes described at A, B, and C, and the other of which includes the processes described at A and C. The methods depicted in FIG. 4 generally include introducing the dendron architecture 26 to the surface of the substrate 16, wherein the dendron architecture 26 includes: the functionalized focal point of attachment 28 that attaches to the substrate surface, and the plurality of peripheral functional groups 30 that are orthogonal to the functionalized focal point of attachment 28.

It is to be understood that the example methods shown in FIG. 4 may be performed with or without the base support 14 (e.g., upon which the resin material of the resin layer 18 may be applied and cured). As such, the method processes described below regarding the resin layer 18 are also applicable to a single-layer version of the substrate 16.

Any suitable resin material described herein may be used to form the resin layer 18. Further, any suitable deposition technique disclosed herein may be used to deposit the resin (that forms the resin layer 18) on the base support 14. Still further, the base support 14 may be any of the base support materials described herein.

After the resin layer 18 has been applied to the base support 14, the depression(s) 20 and interstitial regions 22 or the lane 34 and bonding region 38 are defined in the resin layer 18 as described in reference to the FIG. 2 series. While a single depression 20 or lane 34 is shown in the method depicted in FIG. 4A through FIG. 4C, it is to be understood that the flow cell 10 may include a plurality of depressions 20, where each individual depression 20 is separated from each other depression 20 by interstitial regions 22 (similar to that shown in FIG. 1C and FIG. 1D). FIG. 4A specifically illustrates the release of the working stamp 40 and the formation of the resin layer 18. In this particular example, the release of the working stamp 40 from the resin layer 18 creates the depression 20 or lane 34 in the layer 18.

Following the formation of the depression(s) 20 or the lane 34 in the resin layer 18, the method may then proceed to either of B or C. This is because, in some instances, the primer set 32 is pre-grafted to the plurality of peripheral functional groups 30; and, in other instances, the method involves grafting the primer set 32 to the plurality of peripheral functional groups 30 (e.g., after the dendron 26 has been anchored to the resin layer 18). As such, whether the method proceeds from A to B or from A to C will depend upon whether the primers of the primer set 32 have been pre-grafted to the peripheral groups 30 of the dendron architecture 26. When the primers are pre-grafted to the dendron 26, the method proceeds from A to C. When the primers are not pre-grafted to the dendron 26, the method proceeds from A to B.

As described, when the dendron architecture 26 is not pre-grafted with the primers of the primer set 32, the method proceeds by introducing the (non-pre-grafted) dendron 26 to the resin layer 18. This is shown in B of FIG. 4. Introduction of the non-pre-grafted dendron 26 to the resin layer 18 may be accomplished using any suitable technique as described herein. When the dendron architecture 26 comes into contact with the resin layer 18, the focal point 28 interacts (e.g., forms a chemical bond) with functional groups of the resin layer 18, thereby forming an attachment point and anchoring the dendron 26 to the resin layer 18. This process leaves the peripheral groups 30 of the dendron architecture chemically available for subsequent primer attachment.

While the dendron architecture 26 depicted in B includes four visible peripheral groups 30, it is to be understood that other amounts of peripheral groups 30 may be included in the dendron 26. In an example, the number of peripheral groups 30 included in the dendron architecture ranges from 2 to 20. The peripheral groups 30 and the focal point 28 may include any of the chemical functionalities described herein (in regard to Table 1 and Table 2). In one specific example, the functionalized focal point of attachment is a tetrazine, the plurality of peripheral functional groups 30 is a plurality of azides, and the substrate surface includes a norbornene functional group that attaches the functionalized focal point of attachment 28 to the substrate surface.

In some instances, the method further includes activating the resin layer 18 as described herein. In one example, the method involves silanizing the substrate surface prior to introducing the dendron architecture 26 thereto. Activation of the substrate surface may be accomplished using a silanizing solution or using plasma ashing.

Proceeding from B to C in FIG. 4, following the introduction of the non-pre-grafted dendron 26 to the resin layer 18, the method involves grafting a primer set 32 (including two different primers) to the peripheral groups 30 of the dendron 26 (e.g., after the dendron 26 has been introduced to the substrate surface). Grafting of the primers to the peripheral groups 30 may be accomplished using any suitable grafting technique. As examples, grafting of the primers to the peripheral groups 30 may be accomplished by flow through deposition (e.g., using a temporarily bound lid), dunk coating, spray coating, puddle dispensing, or by another suitable method. Each of these example techniques may utilize a primer solution or mixture, which includes the primers of the primer set 32, water, a buffer, and a catalyst. It is to be understood that the buffer and the catalyst, as well as other additives, may or may not be included in the primer solution or mixture during the incubation process depending upon the coupling reaction that is to take place. With any of the grafting methods, the primers of the primer set 32 attach to the reactive peripheral groups 30 of the dendron 26, and have no affinity for any unreacted focal points 28.

When the dendron 26 is pre-grafted with the primers of the primer set 32, it is to be understood that the method proceeds directly from A to C in FIG. 4. In these examples, the primers of the primer set 32 are already attached to the peripheral groups 30 of the dendron 26 when the dendron 26 is introduced to the substrate surface. The focal point 28 of the dendron 26 interacts (e.g., bonds with) the resin layer 18, thereby anchoring the pre-grafted dendron 26 to the layer 18.

In some instances, the method further includes silanizing (or otherwise activating) the surface of the substrate 16 (e.g., the resin layer 18) prior to introducing the pre-grafted dendron architecture 26 to the surface of the substrate 16. Selective application of the depression(s) or lane 34 may take place so that the dendrons 26 attach to the depression(s) or lane 34 and not to the interstitial regions 22 or bonding region 38.

Alternatively, the interstitial regions 22 or bonding regions 38 may be masked while the dendrons 26 are applied within the depression(s) or lane 34. In still other examples, any dendrons 26 attached at the interstitial regions 22 or bonding regions 38 may be removed via polishing.

Regardless of whether the dendron 26 is pre-grafted with primers of the primer set 32, the patterned or unpatterned structure that is formed and that is shown in C of FIG. 4 can then be used in an open wafer sequencing process or bonded to the lid or to another patterned structure using the spacer layer and a suitable bonding method as described herein.

While not shown in FIG. 4 (A through C), in some examples, prior to introducing the (pre-grafted or non-pre-grafted) dendron architecture 26 to the substrate surface, the method further comprises grafting a regenerating moiety 42 (see FIG. 1D) to the substrate surface, and the focal point 28 attaches to the regenerating moiety 42. The regenerating moiety 42 used in these examples (and the reagent used to reversibly decouple the regenerating moiety 42) may be any suitable example described herein.

To further illustrate the present disclosure, examples are given herein. It is to be understood that these examples are provided for illustrative purposes and are not to be construed as limiting the scope of the present disclosure.

### NON-LIMITING WORKING EXAMPLES

### Example 1

In this example, two different patterned substrates, each including a norbornene silanized resin material patterned with depressions in eight different lanes, was used to test the stability of two different dendrons: one having formula (I) as shown herein (referred to in this example as “MPA-Tz₄N₃”) and the other having formula (II):

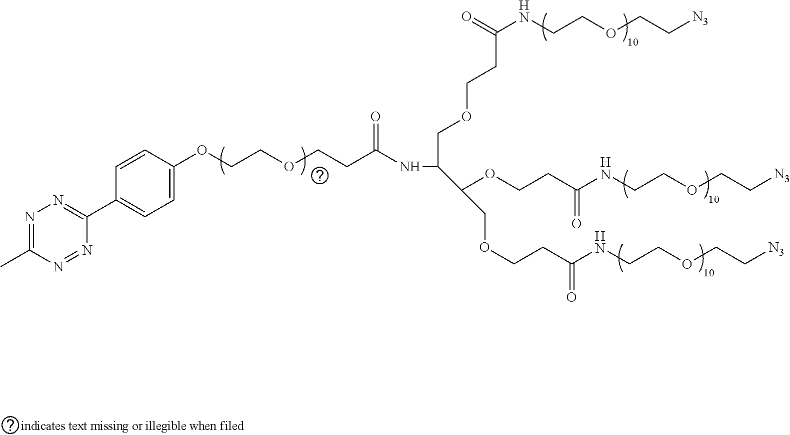

(referred to in this example as “PEG-Tz3N₃”). The first of the dendrons was attached in the eight lanes of one of the patterned substrates, and the second of the dendrons was attached in the eight lanes of the other of the patterned substrates by incubating each lane in a 1 mM solution of the respective dendron in ethanol for about 1 hour at room temperature.

Each of the lanes of the patterned substrates was incubated in a different solution to expose the dendrons to different aqueous stresses (some of which are used in sequencing by synthesis). The following protocol was utilized for incubation: lanes 1 and 8=no solution (i.e., without stress), lanes 2 and 3=water (i.e., H₂O), lanes 4 and 5=a carbonate buffer, lanes 6 and 7=aqueous sodium sulfate (i.e., Na₂SO₄), and solution exposure for 1 hour at 60° C. The incubation solutions were removed, and 1 μM of a DBCO-Cy3 fluorophore was introduced into each lane in order to probe the presence of the azides on the dendrons. The fluorescent intensity was measured in an Amersham TYPHOON™ scanner. The results are depicted in FIG. 5 where the flow cell lanes are identified by the solution protocol used. Overall, both dendron types exhibited suitable stability, at least in the conditions of no solution, water, and aqueous sodium sulfate. When comparing the lanes containing the same solution across the two different flow cells, the MPA-Tz4N₃ dendrons displayed higher normalized median intensity than the PEG-Tz3N₃ dendrons without stress, when exposed to water, and when exposed to aqueous sodium sulfate, thus exhibiting better stability.

### Example 2

Patterned substrates as described in Example 1 were used in this example.

One of the patterned substrates was used as a control, where tetrazine terminated P5 and P7 primers (in a 5 μM solution) were grafted directly to the norbornene silane surface before a lid was bonded.

MPA-Tz4N₃ dendrons were introduced into different lanes of the other of the patterned substrates as described in Example 1. This patterned substrate was polished and bonded to a lid. Bicyclononyne terminated P5 and P7 primers were grafted to the previously attached MPA-Tz4N₃ dendrons at different concentrations, namely 5 μM, 8 μM, 10 μM, and 15 μM. Grafting took place in 0.9 M Na₂SO₄ for about 2 hours at 60° C. This was reproduced to generate two example flow cells.

For comparison, a commercially available HISEQX™ flow cell (a patterned substrate including a hydrogel and primers) was also used.

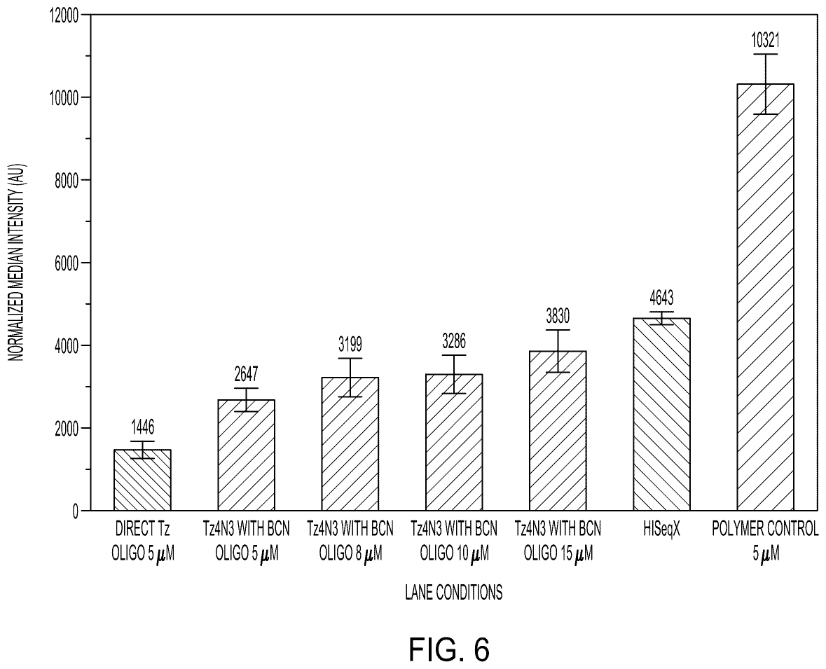

Each of these flow cells (including one of the example flow cells) was exposed to a quality control test using CAL FLUOR RED™ (CFR) labeled complements of the P5 and P7 primers. The fluorescence intensity was measured in an Amersham TYPHOON™ scanner. The results are depicted in FIG. 6. As can be seen, the flow cell including the MPA-Ta4N₃ dendron and P5 and P7 primers displayed higher normalized median intensity, relative to the flow cell that did not include the intervening MPA-Ta4N₃ dendron structure. These results indicate that the MPA-Ta4N₃ dendron is capable of controlling primer concentration in a flow cell, relative to surface area, to increase the number of grafted primers and thus potentially increasing sequencing metrics. Following the quality control test, the flow cells were exposed to 0.1 M NaOH for dehybridization of the CFR labeled complements.

The example flow cell exposed to the quality control test and the other of the example flow cells not exposed to the quality control test were used to assess clustering efficiency. Library fragments (from the PhiX genome) were introduced and clustering was performed using bridge amplification. After clustering, fluorescence images were taken (with the sequencer) of different tiles within the lanes including grafted BCN terminated P5 and P7 primers (at 15 μM). While not reproduced herein, the images of the example flow cell that was not exposed to the quality control test illustrated successful cluster generation, while the images of the example flow cell exposed to the quality control test illustrated sparser clusters. The sparse clusters were likely due to the exposure to the base following the quality control test.

### Example 3

This example compared dendritic primers and linear primers.

The dendritic primers included azide terminated P5 and/or P7 primers attached to a 3-arm dendron including an alkyne focal point and dibenzocyclooctyne (DBCO) peripheral groups. The dendritic primers were generated before being introduced to a flow cell surface. Formula (III) represents the structure of the dendritic primers that were generated:

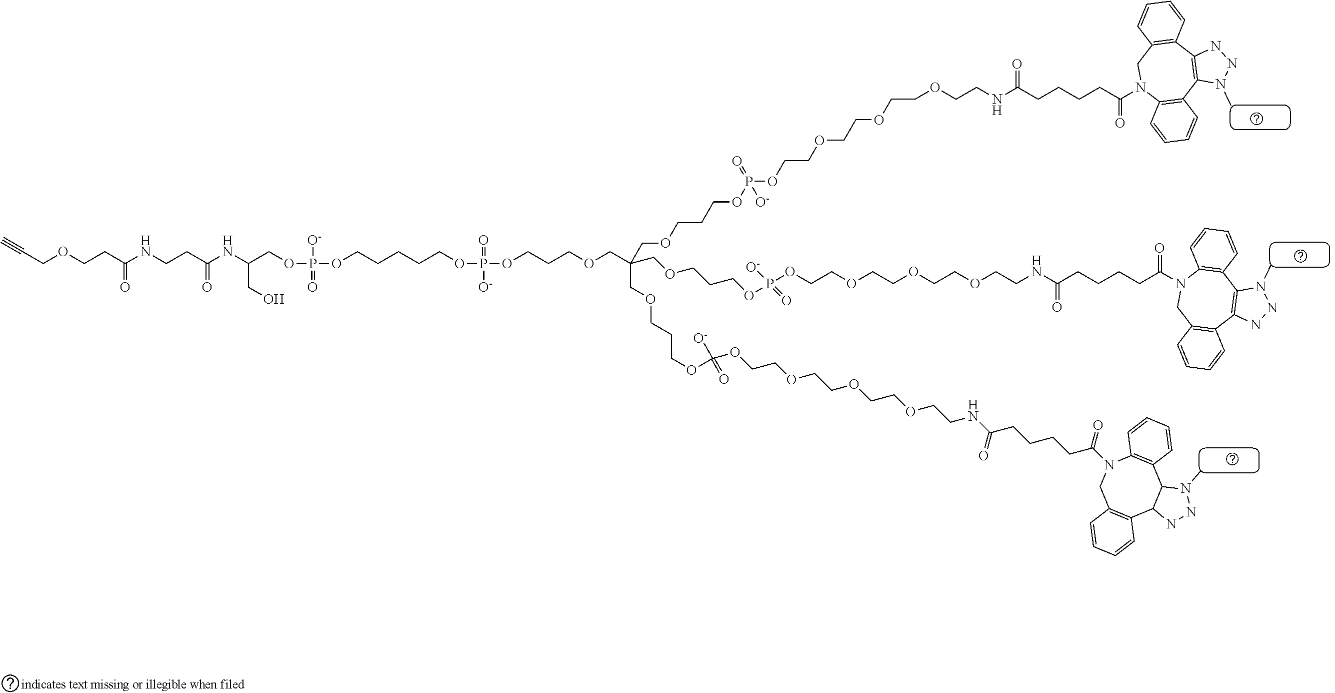

These dendritic primers may be referred to as “Trebler P5/P7 primers” in this example.

The linear primers were P5 and/or P7 primers with a poly T (6T) spacer. These linear primers may be referred to as “T6” or “T6 primers” in this example.

The flow cells that were used in this example included a resin patterned with depressions, and a hydrogel attached within the depressions. Different flow cells or different lanes of the same flow cell were used for the Trebler P5/P7 primers and for the T6 primers.

Different concentrations of the Trebler P5/P7 primers (0.4 μM, 0.5 μM, 1 μM, and 1.5 μM) and of the T6 primers (1.2 μM, 1.5 μM, 3 μM, and 4.5 μM) were grafted to the hydrogels in different lanes of the same flow cell or in different lanes of different flow cells using Cu(I)-catalyzed azide-alkyne cycloaddition (CuAAC) reactions.

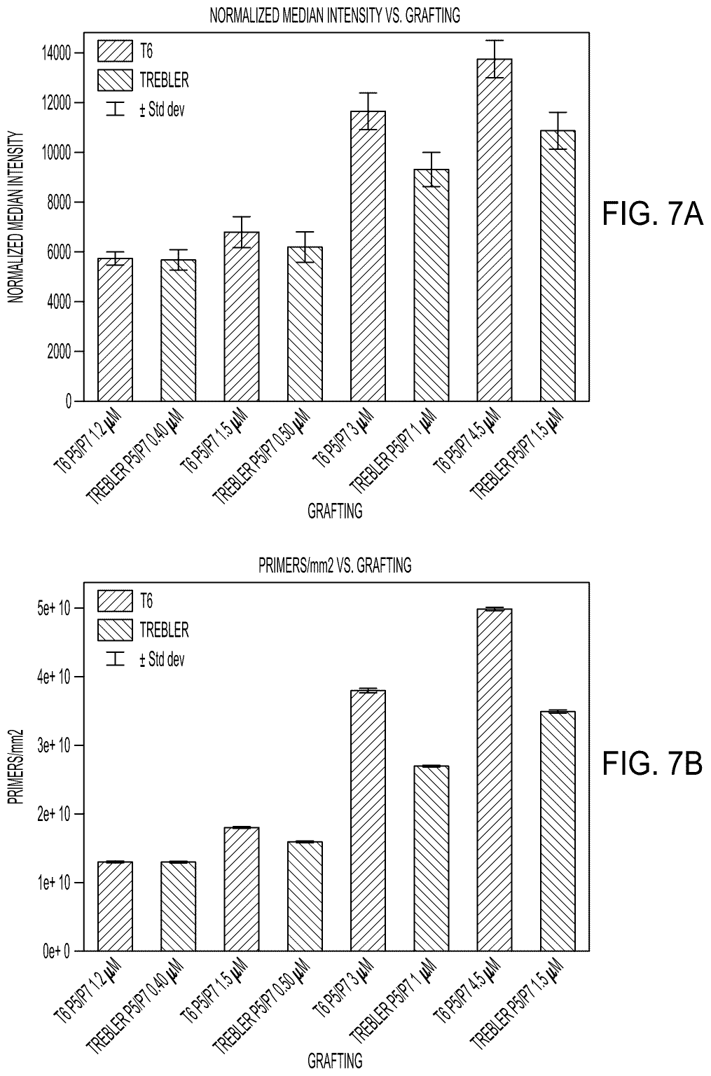

One flow cell (with different primers and/or concentrations in each lane) was exposed to a quality control test using CFR labeled complements of the P5 and P7 portions of the dendritic and linear primers. The fluorescence intensity was measured in an Amersham TYPHOON™ scanner. The normalized median intensity for each lane (identified by the type and concentration of the grafted primer) is shown in FIG. 7A. The primer density was determined from the fluorescence intensity, and these results are shown in FIG. 7B.

The results in FIG. 7A and FIG. 7B demonstrate that the Trebler P5/P7 primers achieved similar CFR intensities and similar primer densities as the T6 primers at ⅓ of the grafting concentration (e.g., 1.2 μM for the T6 primers and 0.4 μM for the Trebler P5/P7 primers, or 1.5 μM for the T6 primers and 0.5 M for the Trebler P5/P7 primers). These results are consistent with the presence of three P5/P7 primers per dendron. These results also indicate that the click reaction to the surface and the hybridization of complementary P5 and P7 primers work as efficiently for the Trebler P5/P7 primers as for the linear T6 primers under similar grafting concentration ranges (<2 μM).

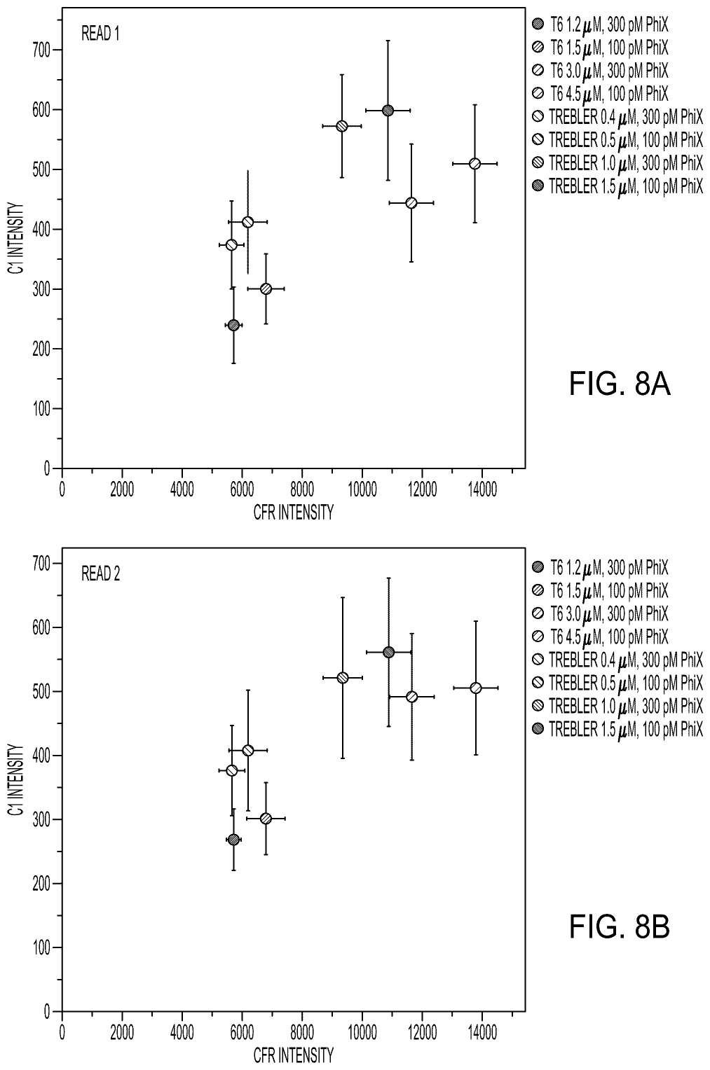

One flow cell (with different primers and/or concentrations in each lane) was exposed to several sequencing cycles. Different concentrations of library fragments (from the PhiX genome) were introduced and clustering was performed using Illumina Inc.'s EXAMP™ workflow. After clustering, sequencing-by-synthesis (SBS, 2×26 cycles) was performed. The read 1 (R1) fluorescence intensity and the read 2 (R2) fluorescence intensities for the red channel after one sequencing cycle (C1) are plotted in FIG. 8A and FIG. 8B as a function of the CFR intensity (y axis, from FIG. 7A).

As depicted in FIG. 8A and FIG. 8B, functionalization with Trebler P5/P7 primers led to an increase (up to 60%) in C1 intensity during SBS compared to the T6 primers at equivalent primer densities for both reads. For example, lanes grafted with the Trebler P5/P7 primers at 0.4 μM showed a 1.6× increase in C1 intensity compared to lanes grafted with T6 primers at 1.2 μM for read 1 and a 1.4× increase for read 2. Given the nearly matched primer densities, the increase in C1 intensity may be due to the branched surface architecture, which may provide better accessibility to strands within clusters during SBS.

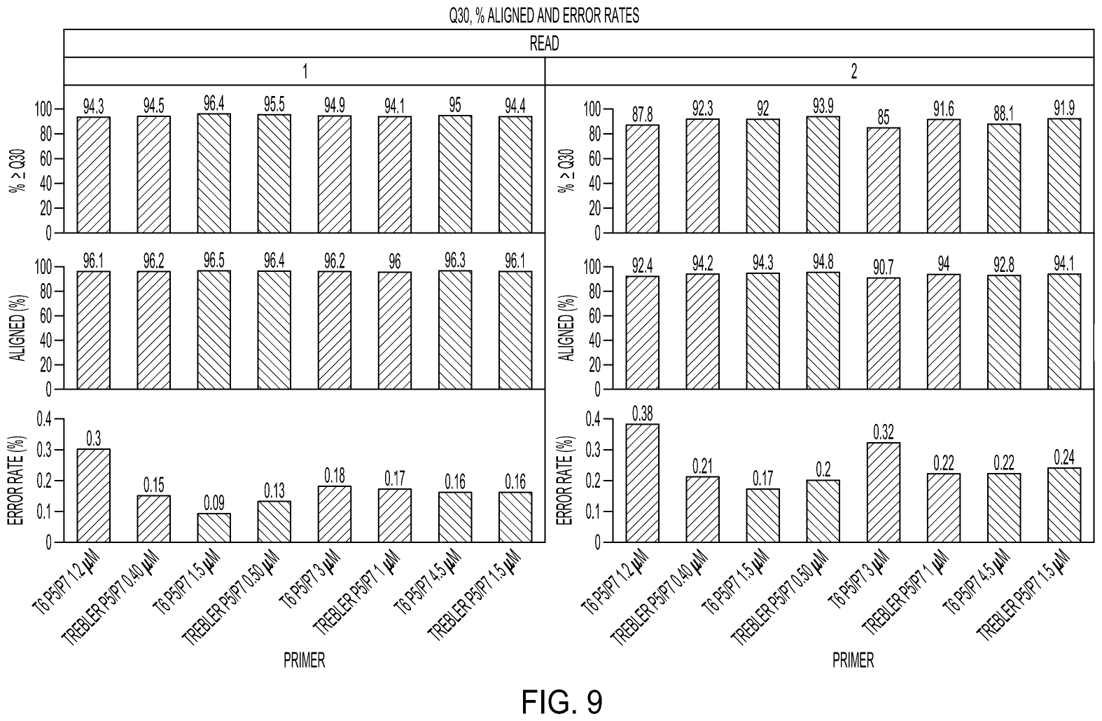

The sequencing data collected included error rate (%) (percentage), aligned (%), and Q30. The error rate represents the percentage of incorrect base calls against the PhiX genome. The aligned percentage refers to the percentage of reads that are aligned to the reference genome. A higher aligned percentage is indicative of the accuracy of the sequencing. Q30 (%) is the percentage of Qscores that were greater than Q30. A Qscore of 30 (Q30) is equivalent to the probability of an incorrect base call 1 in 1000 times. This means that the base call accuracy (i.e., the probability of a correct base call) is 99.9%. This means that the base call accuracy (i.e., the probability of a correct base call) is 99.9%. A lower base call accuracy of 99% (Q20) will have an incorrect base call probability of 1 in 100, meaning that every 100 base pair sequencing read will likely contain an error. When sequencing quality reaches Q30, virtually all of the reads will be perfect, having 99.9% accuracy. All of these results are shown in FIG. 9 for the library concentration used and as a function of the primers grafted.

The error rate, % aligned, and %>Q30 results for the flow cells with Trebler P5/P7 primers were similar to those with T6 primers.

Another flow cell (with 0.4 μM Trebler P5/P7 primers in four lanes and 1.2 μM T6 primers in four other lanes) were tested on a 2-channel HISEQX™ sequencer (Illumina, Inc.) using fully functional nucleotides for a 2-channel system. The primary metrics for a 1×150 cycle run are shown in Table 4. The Trebler P5/P7 primers again showed a 1.6× to a 1.9× higher C1 intensity than the linear T6 primers at similar densities on the surface and similar library inputs.

TABLE 4

| | | CFR | | | | | | | | |
|---|---|---|---|---|---|---|---|---|---|---|
| | | Intensity | PhiX | | | Pre- | % >= | Aligned | Error | C1 |
| Lane | Primer | (x1000) | (pM) | PF (%) | Phasing | phasing | Q30 | (%) | Rate (%) | Intensity |
| 1 | 1.2 μM | 4.7 ± 0.5 | 150 | 58.28 ± 3.87 | 0.046/1.339 | 0.092/0.313 | 83.63 | 92.75 ± 4.94 | 5.20 ± 4.33 | 592 ± 67 |
| | T6 | | | | | | | | | |
| 2 | 1.2 μM | 5.1 ± 0.5 | 100 | 57.99 ± 6.83 | 0.046/1.265 | 0.082/0.703 | 85.05 | 93.77 ± 4.28 | 3.99 ± 3.02 | 590 ± 57 |
| | T6 | | | | | | | | | |
| 3 | 1.2 μM | 5.7 ± 0.4 | 75 | 48.08 ± 5.57 | 0.045/1.207 | 0.100/0.164 | 85.07 | 93.87 ± 3.96 | 4.59 ± 3.51 | 527 ± 32 |
| | T6 | | | | | | | | | |
| 4 | 1.2 μM | 5.9 ± 0.6 | 50 | 49.02 ± 3.75 | 0.045/1.219 | 0.098/0.158 | 85.26 | 93.97 ± 4.28 | 4.55 ± 3.69 | 530 ± 27 |
| | T6 | | | | | | | | | |
| 5 | 0.4 μM | 6.2 ± 0.4 | 150 | 52.15 ± 3.84 | 0.056/1.373 | 0.096/0.428 | 82.57 | 92.64 ± 7.61 | 4.66 ± 3.80 | 977 ± 71 |
| | Trebler | | | | | | | | | |
| 6 | 0.4 μM | 6.3 ± 0.4 | 100 | 54.27 ± 5.89 | 0.058/1.192 | 0.079/0.901 | 84.31 | 92.43 ± 10.14 | 4.28 ± 3.89 | 1102 ± 89 |
| | Trebler | | | | | | | | | |
| 7 | 0.4 μM | 5.7 ± 0.4 | 75 | 53.14 ± 4.60 | 0.054/1.460 | 0.065/1.731 | 85.37 | 93.09 ± 4.40 | 3.70 ± 2.61 | 976 ± 77 |
| | Trebler | | | | | | | | | |
| 8 | 0.4 μM | 5.4 ± 0.5 | 50 | 49.99 ± 11.08 | 0.053/1.206 | 0.074/1.289 | 84.26 | 89.27 ± 16.83 | 4.16 ± 3.24 | 938 ± 171 |
| | Trebler | | | | | | | | | |

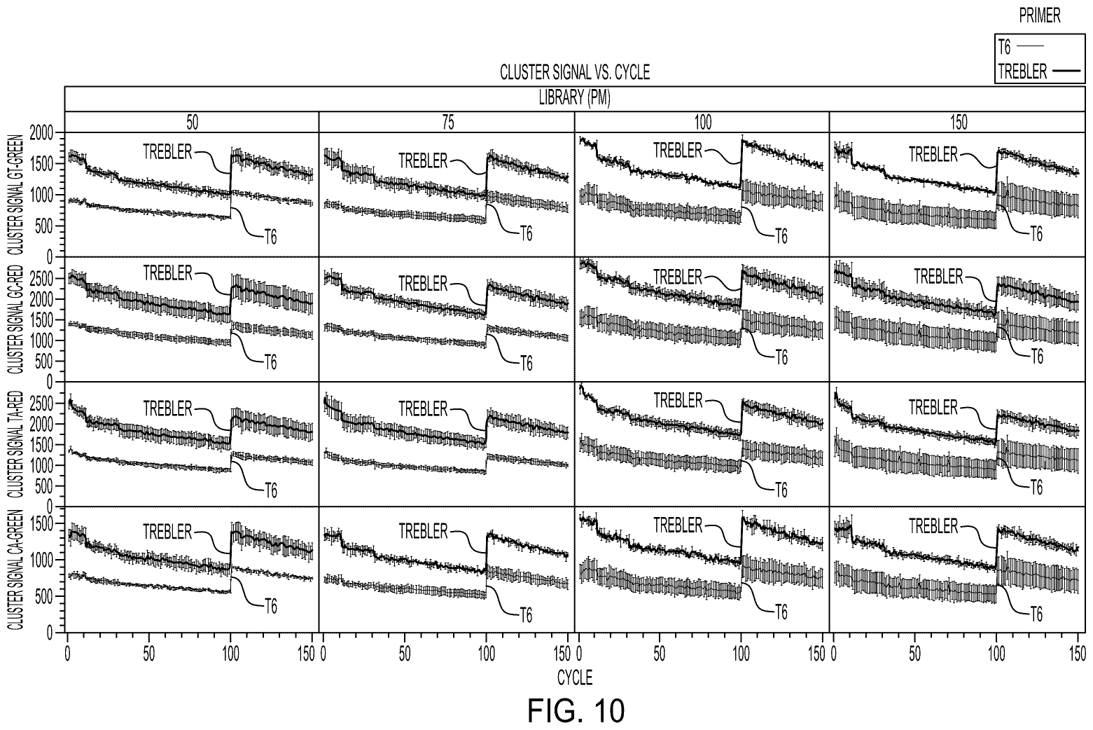

Offline analysis was used to extract the cluster signal metrics, where cluster signal is defined as the difference between the mean raw intensity of the ON state and the mean raw intensity of the OFF state for a given base pairing. As shown in FIG. 10, the Trebler P5/P7 primers showed higher cluster signal for all base pairings under the tested conditions, which was maintained in the later cycles.

### Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

While several examples have been described in detail, it is to be understood that the disclosed examples may be modified. Therefore, the foregoing description is to be considered non-limiting.

What is claimed is:

1. A flow cell, comprising:
a substrate including a surface;
a dendron architecture including:
a functionalized focal point of attachment that is attached to the substrate surface; and
a plurality of peripheral functional groups that are orthogonal to the functionalized focal point of attachment; and
a primer set attached to the dendron architecture via the plurality of peripheral functional groups.

2. The flow cell as defined in claim 1, wherein the substrate surface includes a silane that attaches the functionalized focal point of attachment to the substrate surface.

3. The flow cell as defined in claim 1, wherein the substrate surface is free of silane.

4. The flow cell as defined in claim 1, wherein the substrate surface includes a regenerating moiety, and wherein the functionalized focal point of attachment is attached to the substrate surface via bonding with the regenerating moiety.

5. The flow cell as defined in claim 1, wherein:
the functionalized focal point of attachment includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate; and
each of the plurality of peripheral functional groups includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate.

6. The flow cell as defined in claim 1, wherein:
the functionalized focal point of attachment is a tetrazine;
the plurality of peripheral functional groups is a plurality of azides; and
the substrate surface includes a norbornene functional group that attaches the functionalized focal point of attachment to the substrate surface.

7. The flow cell as defined in claim 1, wherein the number of peripheral functional groups included in the dendron architecture ranges from 2 to 20.

8. A method, comprising:
introducing a dendron architecture to a surface of a substrate, wherein the dendron architecture includes:
a functionalized focal point of attachment that attaches to the substrate surface; and
a plurality of peripheral functional groups that are orthogonal to the functionalized focal point of attachment.

9. The method as defined in claim 8, wherein a primer set is pre-grafted to the plurality of peripheral functional groups.

10. The method as defined in claim 8, further comprising grafting a primer set to the plurality of peripheral functional groups.

11. The method as defined in claim 8, wherein prior to introducing the dendron architecture to the substrate surface, the method further comprises grafting a regenerating moiety to the substrate surface, and wherein the functionalized focal point of attachment attaches to the regenerating moiety.

12. The method as defined in claim 8, further comprising silanizing the substrate surface prior to introducing the dendron architecture thereto.

13. The method as defined in claim 8, wherein:
the functionalized focal point of attachment includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate; and
each of the plurality of peripheral functional groups includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate.

14. The method as defined in claim 8, wherein:
the functionalized focal point of attachment is a tetrazine;
the plurality of peripheral functional groups is a plurality of azides; and
the substrate surface includes a norbornene functional group that attaches the functionalized focal point of attachment to the substrate surface.

15. The method as defined in claim 8, wherein the number of peripheral functional groups included in the dendron architecture ranges from 2 to 20.

16. A method, comprising:
depositing a polymeric hydrogel directly over a surface of a substrate; and
introducing a dendron architecture to a surface of the polymeric hydrogel, wherein the dendron architecture includes:
a functionalized focal point of attachment that attaches to the polymeric hydrogel surface,
a plurality of peripheral functional groups that are orthogonal to the functionalized focal point of attachment, and
a primer set grafted to the plurality of peripheral functional groups prior to the introduction of the dendron architecture to the polymeric hydrogel surface.

17. The method as defined in claim 16, wherein prior to introducing the dendron architecture to the polymeric hydrogel surface, the method further comprises grafting a regenerating moiety to the polymeric hydrogel surface, and wherein the functionalized focal point of attachment attaches to the regenerating moiety.

18. The method as defined in claim 16, further comprising silanizing the substrate surface prior to depositing the polymeric hydrogel thereon.

19. The method as defined in claim 16, wherein:
the functionalized focal point of attachment includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate; and
each of the plurality of peripheral functional groups includes a functional group that is selected from the group consisting of an azide, an aryl azide, an amine, a norbornene, a tetrazole, a tetrazine, a sulfonyl fluoride, a thiol, an epoxy, a phosphine having at least two phenyl groups that are capable of undergoing a Staudinger reaction, an acrylate, an alkyne, a cyclooctyne based molecule that is capable of undergoing a strain-promoted alkyne-azide cycloaddition reaction, a cyclooctene based molecule that is capable of undergoing a strain-promoted alkyne-azide cycloaddition reaction or an inverse electron demand Diels Alder reaction, a terminal alkene, an activated ester, and an aryl fluorosulfate.

20. The method as defined in claim 16, wherein:
the functionalized focal point of attachment is an N-hydroxysuccinimide;
the plurality of peripheral functional groups is a plurality of azides; and
the polymeric hydrogel surface includes an amine functional group that attaches the functionalized focal point of attachment to the polymeric hydrogel surface.

21. The method as defined in claim 16, wherein the number of peripheral functional groups included in the dendron architecture ranges from 2 to 20.

22. A dendron architecture represented by a formula (I): [chemical structure]


SEQUENCE LISTING (11 sequences)

SEQ ID NO: 1 - DNA, 29 bp, Synthetic construct
  source 1..29: mol_type = other DNA
  modified_base 23: mod_base = OTHER; note = uracil
aatgatacggcgaccaccgagatctacac

SEQ ID NO: 2 - DNA, 29 bp, Synthetic construct
  source 1..29: mol_type = other DNA
  modified_base 23: mod_base = OTHER; note = inosine
aatgatacggcgaccaccgaganctacac

SEQ ID NO: 3 - DNA, 29 bp, Synthetic construct
  source 1..29: mol_type = other DNA
  modified_base 23: mod_base = OTHER; note = alkene-thymidine
aatgatacggcgaccaccgaganctacac

SEQ ID NO: 4 - DNA, 24 bp, Synthetic construct
  source 1..24: mol_type = other DNA
  modified_base 22: mod_base = OTHER; note = 8-oxoguanine
caagcagaagacggcatacganat

SEQ ID NO: 5 - DNA, 24 bp, Synthetic construct
  source 1..24: mol_type = other DNA
  modified_base 20: mod_base = OTHER; note = 8-oxoguanine
caagcagaagacggcatacnagat

SEQ ID NO: 6 - DNA, 24 bp, Synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 7 - DNA, 29 bp, Synthetic construct
  source 1..29: mol_type = other DNA
  misc_difference 23: note = allyl-T
aatgatacggcgaccaccgaganctacac

SEQ ID NO: 8 - DNA, 35 bp, Synthetic construct
  source 1..35: mol_type = other DNA
gctggcacgtccgaacgcttcgttaatccgttgag

SEQ ID NO: 9 - DNA, 35 bp, Synthetic construct
  source 1..35: mol_type = other DNA
cgtcgtctgccatggcgcttcggtggatatgaact

SEQ ID NO: 10 - DNA, 35 bp, Synthetic construct
  source 1..35: mol_type = other DNA
acggccgctaatatcaacgcgtcgaatccgcaact

SEQ ID NO: 11 - DNA, 34 bp, Synthetic construct
  source 1..34: mol_type = other DNA
gccgcgttacgttagccggactattcgatgcagc